SEQUENCE LISTING (8 sequences)

SEQ ID NO: 1 - DNA, 24 bp, Mus musculus
  source 1..24: mol_type = other DNA
acatctaccacgcagtcaaggacc

SEQ ID NO: 2 - DNA, 24 bp, Mus musculus
  source 1..24: mol_type = other DNA
ctcaagaacatcgcctccattcag

SEQ ID NO: 3 - DNA, 18 bp, Mus musculus
  source 1..18: mol_type = other DNA
gcccgctcgccatctctc

SEQ ID NO: 4 - DNA, 20 bp, Mus musculus
  source 1..20: mol_type = other DNA
gttgagcaggttcccggtct

SEQ ID NO: 5 - DNA, 20 bp, Mus musculus
  source 1..20: mol_type = other DNA
agcgttcggtattacgatcc

SEQ ID NO: 6 - DNA, 22 bp, Mus musculus
  source 1..22: mol_type = other DNA
agtacaatcagggctcttctcg

SEQ ID NO: 7 - DNA, 20 bp, Mus musculus
  source 1..20: mol_type = other DNA
acgcatatacccgctacctg

SEQ ID NO: 8 - DNA, 20 bp, Mus musculus
  source 1..20: mol_type = other DNA
ccagagtgttcattcgagca

(19) United States
(12) Patent Application Publication
(10) Pub. No.: US 2023/0181670 A1
(43) Pub. Date: Jun. 15, 2023

(54) COMPOSITIONS AND METHODS FOR TREATMENT OF ALCOHOL INDUCED LIVER INJURY

(71) Applicant: University of Louisville Research Foundation, Inc., Louisville, KY (US)

(72) Inventor: Huang-Ge Zhang, Louisville, KY (US)

(73) Assignee: University of Louisville Research Foundation, Inc., Louisville, KY (US)

(21) Appl. No.: 18/055,153

(22) Filed: Nov. 14, 2022

Related U.S. Application Data
(62) Division of application No. 16/816,214, filed on Mar. 11, 2020, now abandoned.
Application No. 16/816,214 is a continuation of application No. 15/821,408, filed on Nov. 22, 2017, now abandoned.
(60) Provisional application No. 62/425,320, filed on Nov. 22, 2016.

(51) Int. Cl. A61K 36/9068; A61P 1/16; A61K 31/12; A61K 9/51; A61K 9/14; A61P 1/00; A61K 9/00; A61K 31/26; A61K 36/31

(52) U.S. Cl. CPC A61K 36/9068; A61K 9/14; A61K 9/0053; A61K 9/5123; A61K 9/5176; A61K 31/12; A61K 31/26; A61K 36/31; A61P 1/00; A61P 1/16

(57) ABSTRACT

Methods for treating alcohol induced liver injury include administering to a subject an effective amount of a ginger-derived nanoparticle. Methods for decreasing nuclear factor erythroid-2 related factor (Nrf2) activation in a hepatocyte are also provided and include contacting the hepatocyte with an effective amount of a ginger-derived nanoparticle. Pharmaceutical preparations including ginger-derived nanoparticles are further provided.

### RELATED APPLICATIONS

This application is a divisional of U.S. Pat. Application Serial No. 16/816,214, filed Mar. 11, 2020 (pending), which is a continuation of U.S. Pat. Application Serial No. 15/821,408, filed Nov. 22, 2017 (abandoned), which itself claims priority from U.S. Provisional Application Serial No. 62/425,320, filed Nov. 22, 2016, the entire disclosure of each of which is incorporated herein by this reference.

### GOVERNMENT INTEREST

This invention was made with government support under grant nos. UH2TR000875, RO1AT004294 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 2,786 byte UTF-8-encoded XML file created on Nov. 14, 2022 and entitled “1577_49_4_DIV.xml”. The Sequence Listing submitted via Patent Center is hereby incorporated by reference in its entirety.

### TECHNICAL FIELD

The presently disclosed subject matter relates to compositions and methods for the treatment of alcohol induced liver injury. In particular, the presently disclosed subject matter relates to compositions and methods for the treatment of alcohol induced liver injury that make use of an effective amount of a ginger-derived nanoparticle.

### BACKGROUND

Numerous naturally occurring nanoparticles exist in human diets and are absorbed through the intestine daily. However, whether nanoparticles from plants that are eaten daily by humans can pass from the intestine to the liver, and subsequently, have a biological effect on the liver is poorly defined. Studies have shown that ginger has a hepatoprotective effect against ethanol, carbon tetrachloride, and acetaminophen-induced hepatotoxicity. In this regard, shogaols, dehydrated analogues of the gingerols, have been a focus of in vitro research related to the anti-inflammatory effects of ginger. To date though, the data that has been presented has been data that was derived from using shogaol enriched ginger extract. The biological effect of shogaols in the context of ginger has not been investigated.

The liver itself receives numerous and varied biological insults daily, including alcohol induced liver injuries. The induction of cytoprotective enzymes, including antioxidant and carcinogen-detoxification enzymes, is important for maintaining hepatic homeostasis, and preventing injury from absorbed endotoxin. Nuclear factor erythroid 2-related factor 2 (Nrf2) transcriptionally controls the gene expression of many of those cytoprotective enzymes and plays an important role in protecting the liver against insults. Accordingly, a composition that, unlike the free form of shogaols, can be target-delivered to a liver of a subject and provide for the cytoprotection of liver cells would be both highly desirable and beneficial.

### SUMMARY

The presently disclosed subject matter meets some or all of the above-identified needs, as will become evident to those of ordinary skill in the art after a study of information provided in this document.

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this summary does not list or suggest all possible combinations of such features.

The presently disclosed subject matter includes compositions and methods for the treatment of alcohol induced liver injury. In particular, the presently disclosed subject matter includes compositions and methods for the treatment of alcohol induced liver injury that make use of an effective amount of a ginger-derived nanoparticle.

In some embodiments, a method of treating an alcohol induced liver injury is provided that comprises the step of administering to a subject an effective amount of a ginger-derived nanoparticle. In some embodiments, the ginger-derived nanoparticle is administered orally. In some embodiments, the ginger-derived nanoparticles are administered prior to contacting a liver cell of a subject with an amount of alcohol.

With respect to the ginger-derived nanoparticles of the presently disclosed subject matter, in some embodiments, the ginger-derived nanoparticles have an average diameter of about 100 nm to about 1000 nm such as, in some embodiments, an average diameter of about 300 nm to about 400 nm. In some embodiments, the ginger-derived nanoparticle is comprised of a phosphatidic acid (PA), a digalactosyldiacylglycerol (DGDG), a monogalactosyl monoacylglycerol (MGMG), and combinations thereof. For example in some embodiments, the ginger-derived nanoparticles are comprised of about 30% to about 40% PA, about 30% to about 40% DGDG, and about 10% to about 20% MGMG. In some embodiments, the ginger-derived nanoparticle includes an effective amount of a shogaol.

In some embodiments, administration of a ginger-derived nanoparticle increases a protective effect in the liver of a subject and/or reduces one or more symptoms of liver injury in a subject. For instance, in some embodiments, administering the ginger-derived nanoparticles increases an amount of nuclear factor erythroid-2 related factor (Nrf2) activation. In other embodiments, administering the ginger-derived nanoparticles reduces an amount of reactive oxygen species (ROS) in the liver of the subject, reduces an amount of triglycerides in the liver of the subject, decreases a total weight of the liver of the subject, and/or decreases an amount of lipid droplets in the liver of the subject.

Further provided, in some embodiments, are methods of decreasing nuclear factor erythroid-2 related factor (Nrf2) activation in a hepatocyte. In some embodiments, a method of decreasing nuclear factor erythroid-2 related factor (Nrf2) activation in a hepatocyte is provided that comprises contacting the hepatocyte with an effective amount of a ginger-derived nanoparticle.

Still further provided, in some embodiments, are pharmaceutical compositions comprising a ginger-derived nanoparticle and a pharmaceutically acceptable vehicle, carrier, or excipient.

Further advantages of the presently disclosed subject matter will become evident to those of ordinary skill in the art after a study of the description, figures, and non-limiting examples in this document.

### BRIEF DESCRIPTION OF THE DRAWINGS

FIGS. 1A-1F include images and graphs showing the identification and characterization of ginger derived nanoparticles (GDNs) and ginger-derived exosome-like nanoparticles (GDEN2), including images and graphs showing: (FIG. 1A) two bands from sucrose banded ginger rhizome root derived samples that were formed after gradient ultracentrifugation (left), where GDN and GDEN2 particles were visualized by Atomic Force Microscorpy (AFM); (FIG. 1B) size distribution (left) and surface Zeta-potential (right) of the particles determined using a Zetasizer Nano ZS; (FIG. 1C) a pie chart with a summary of the putative lipid species in GDN and GDEN2, reported as the percentage of total GDENs lipids (PA: phosphatidic acids; PS: phosphatidylserine; PI: phosphatidylinositol; PE: phosphatidylethanolamines; PC: phosphatidylcholine; PG:phosphatidylglycerol; MG/DG: mono/di/glycerols; LysoPG : lysophosphatidylglycerol; LysoPC: lysophosphatidylcholine; LysoPE: lysophosphatidylethanolamines); (FIG. 1D) TLCs (left) and HPLC (right) analysis of the lipid extracts from GDN and GDEN2, where a standard shogaol (left panel, the first lane) or gingerol (left panel, the second lane) were used as markers; (FIG. 1E) TLCs analysis of the lipid extracts from ginger extracts, ginger microparticles, pellet inclduing GDN plus GDEN2, and ginger extracts with GDN and GDEN2 depleted; (FIG. 1F) GDN or GDEN2 incubated in a stomach-like solution (top panel) for 60 min at 37° C. and subsequently in small intestinal-like solution (bottom panel) for an additional 60 min at 37° C., where size distrubtion and surface Zeta-potential changes were measured by Zetasizer Nano ZS.

FIGS. 2A-2E include images and graphs showing the in vivo distrbution of orally administered GDN and GDEN2, inclduing images and graphs showing: (FIG. 2A) in vivo imaging of trafficking of GDN, where male C57BL/6 mice were administered DiR dye labeled GDN (50 mg per mouse in 200 µl PBS) by gavage, and imaged over 24 h (left), and where the results are presented as mean of the net intensity (Sum Intensity/Area, n=5) (right); (FIG. 2B) hepatocytes taking up DIR labeled GDN, where male C57BL/6 mice were gavage administered DIR-GDN, DIR-GDEN2 or grapefruit nanoparticles (GFN) (50 mg per mouse in 200 µl PBS), where, 6h after the administration, frozen sections of liver were examined by confocal microscopy for DIR⁺/Albumin⁺F4/80⁺ cells (left) and were quantified (right), where the original magnification was ×40; confocal image analysis of frozen sections of intestines were from mice fed PKH26⁺GDN or PKH26⁺GDEN2 after immunofluorescent staining for CD31 (FIG. 2C) or lyve-1 (FIG. 2D) (green), where the original magnification ×60 (left panel) with enlargement of indicated area shown in the right panel; (FIG. 2E) blocking of primary hepatocyte uptake of PKH26 labeled GDN, where primary hepatocytes cells were incubated with the indicated chemical reagents or PBS as a control in the presence of PKH26 labeled PKH26⁺GDN or PKH26⁺GDEN2 (100 µg/ml) for 3 h, where the treated cells were then washed, fixed, and cells were stained with anti-mouse albumin, and where PKH26⁺Albumin⁺ cells were examined using confocal microscopy and photographed.

FIGS. 3A-3G include images and graphs showing that TLF4/TRIF regulates GDN shogaol-mediated induction of nuclear transloction of Nrf2 in primary mouse hepatocytes, including images and graphs showing: (FIG. 3A) primary mouse hepatocytes from C57BL/6j mice that were cultured in the presence of GDN or GDENs (100 µg/ml) for 4 hrs, where cells were then fixed and stained with anti-Nrf2 antibody, where Nrf2⁺ cells were examined using confocal microscopy (left) and were quantified (right), with the original magnification being x40, and with an example of Nrf2 translocated from the cytoplasm to the nucleus indicated by white arrows, where the data (right panel) were expressed as fold changes of ratios of the intensity of nuclear to cytoplasmic signal of Nrf2 in cells (*P < 0.05, Student’s t-test); (FIG. 3B) primary mouse hepatocytes from C57BL/6j mice that were cultured for 24 hr in the presence of GDN or GDENs (100 µg/ml), where the induction of ROS at different time points as inducated in FIG. 4B was measured (*P < 0.05, Student’s t-test); (FIG. 3C) lipids extracted from GDN derived liposome-like nanoparticles (LN) or LN with Shogaol knock-out or knock-in and a standard Shogaol that were separated on a thin-layer chromatography plate and developed, where a representative image was scanned using an Odyssey Scanner; (FIG. 3D) primary mouse hepatocytes were cultured for 4 hr in the presence of GDN (d, 100 µg/ml) or LN from GDN (100 µg/ml) with Shogaol knock out or knock in (FIG. 3E), where cytoplasmic and nuclear extracts were isolated, subjected to Western blotting and probed with anti-Nrf2 antibody, or GAPDH or PCNA antibodies as controls (*P < 0.05, **P < 0.01, Student’s t-test); (FIG. 3F) primary mouse hepatocytes cultured for 4 hr in the presence of LN or LN derived from GDN (100 µg/ml) with Shogaol knock out or knock in (Shogaol, 3.18 µM), where the induction of DCF in the treated cells was FACS analyzed, where the data were expressed as fold changes of ratios of the intensity of nuclear to cytoplasmic of Nrf2 in cells; and (FIG. 3G) primary mouse hepatocytes from TLR4, TRIF, and Myd88 knock out mice that were cultured for 4 hr in the presence of GDN (100 µg/ml) or LN from GDN (100 µg/ml) or LN with Shogaol knock out or knock in (Shogaol, 3.18 µM), where cytoplasmic and nuclear extracts were isolated, subjected to western blotting and probed with anti-Nrf2 antibody with GAPDH or PCNA antibodies having served as controls, where the data (bottom panels) were expressed as fold changes of ratios of the intensity of nuclear to cytoplasmic signal of Nrf2 in cells (*P < 0.05, Student’s t-test).

FIGS. 4A-4I include graphs and images showing that oral administration of GDN protects against alcohol induced liver injury, including graphs and images showing: (FIG. 4A) real-time analysis of expression of different genes at 6 hr in the livers of C57BL/6j male mice orally administered with GDN (50 mg in 200 µl PBS) *P < 0.05, (Student’s t-test); (FIG. 4B) male C57BL/6 mice that were intravenously (b, 1st panel from left, 10 µg/mouse) or orally administered (b, 2nd, and 3rd panels, 250 µg per mouse in 200 µl PBS) with IRDye-700DX covalent conjugated GDN, and plasma were collected over 360 min, and scanned with a Li-CoR Scanner, where the amount of GDN in plasma was calculated based on the standard curve made from IRDye-700DX labeled GDN (b, right panel), and where the results are presented as mean of net intensity (Sum Intensity/Area, n=5); (FIG. 4C) quantification of GDN in the plasma, where male C57BL/6 mice were orally administered (250 µg per mouse in 200 µl PBS) IRDye-700DX covalent conjugated and DIR dye labeled GDN or PBS as a control, where plasma was collected 45 min after oral administration, and diluted in PBS at 1:10 for ultracentrifugation at 150,000 g for 2 hr, where the pellets were resuspended in PBS and scanned with a Li-CoR Scanner with representative images being shown (left panel), and where fold changes of intensity of fluorescent signal of GDN versus PBS fed mice were calculated; (FIG. 4D) male C57BL/6 mice were fed with either regular diet (top and middle panels) or a liquid diet containing 5% ethanol daily (bottom panel) for 7 days, where mice fed with regular diet or ethanol diet were orally administered IRDye-700DX covalent conjugated and PKH67 double labeled GDN (250 µg per mouse in 200 µl PBS), where 12 h after the administration, frozen sections of liver were examined by confocal microscopy for PKH67⁺/IRDye-700DX⁺ cells, and where the original magnification is ×40 (first 4 columns from left) with enlargement of indicated area shown in the last columns; (FIGS. 4E-4I) 8-week-old male C57BL/6j mice fed a liquid diet containing 5% ethanol daily for 7 days, where, starting on day 7, mice were gavage-administered GDN (50 mg/day) or PBS as a control daily while continuing the feeding of the 5% ethanol diet until day 14, where at day 14 mice were fed with 30% instead of 5% ethanol and gavaged with a last dose of GDN at 9 hr post ethanol administration, where the mice were euthanized and assessed for (FIG. 4E) levels of ALT and AST in serum, (FIG. 4F) neutral triglycerides and lipids using Oil red stain, (FIG. 4G) liver triglyceride (TG), (FIG. 4H) ratios of liver/body weight, and (FIG. 4I) H&E-stained sections of livers from mice pretreated with PBS or GDN, original magnification ×20 (*P < 0.05 Student’s t-test).

FIGS. 5A-5B are images showing the uptake of GDNs (FIG. 5A) and GDEN2 (FIG. 5B) by primary hepatocytes following treatment with endocytosis inhibitors.

FIG. 6 includes images showing uptake of GDNs was a temperature dependent process.

FIG. 7 includes images and graphs showing a knock-out of shogaol had no effect on the range of sizes of nanoparticles, but provided an additional subpopulation of particles.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently disclosed subject matter.

Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently disclosed subject matter is based, at least in part, on the discovery that, unlike the free form of shogaols, shogaols carried by ginger derived nanoparticles (GDN) can be target-delivered to hepatocytes and can be used to treat alcohol-induced liver injury. The presently disclosed subject matter thus includes compositions and methods for the treatment of alcohol induced liver injury. In particular, the presently disclosed subject matter includes compositions and methods for the treatment of alcohol induced liver injury that make use of an effective amount of a ginger-derived nanoparticle.

The term “nanoparticles” as used herein in reference to the broccoli-derived nanoparticles of the presently disclosed subject matter, refers to nanoparticles that are in the form of small assemblies of lipid particles, are about 50 to 1000 nm in size, and are not only secreted by many types of in vitro cell cultures and in vivo cells, but are also commonly found in vivo in body fluids, such as blood, urine and malignant ascites. Indeed, such nanoparticles include, but are not limited to, particles such as microvesicles, exosomes, epididimosomes, argosomes, exosome-like vesicles, microparticles, promininosomes, prostasomes, dexosomes, texosomes, dex, tex, archeosomes, and oncosomes.

Such nanoparticles can be formed by a variety of processes, including the release of apoptotic bodies, the budding of microvesicles directly from the cytoplasmic membrane of a cell, and exocytosis from multivesicular bodies. For example, exosomes are commonly formed by their secretion from the endosomal membrane compartments of cells as a consequence of the fusion of multivesicular bodies with the plasma membrane. The multivesicular bodies are formed by inward budding from the endosomal membrane and subsequent pinching off of small vesicles into the luminal space. The internal vesicles present in the multivesicular bodies are then released into the extracellular fluid as so-called exosomes.

As part of the formation and release of nanoparticles, unwanted molecules are eliminated from cells. However, cytosolic and plasma membrane proteins are also incorporated during these processes into the microvesicles, resulting in microvesicles having particle size properties, lipid bilayer functional properties, and other unique functional properties that allow the nanoparticles to potentially function as effective nanoparticle carriers of therapeutic agents. In this regard, in some embodiments, the term “nanoparticle” is used interchangeably herein with the terms “microvesicle,” “liposome,” “exosome,” “exosome-like particle,” “nano-vector” and grammatical variations of each of the foregoing.

The phrase “derived from ginger” or “ginger-derived” when used in the context of a nanoparticle, refers to a nanoparticle that, by the hand of man, exists apart from its native environment and is therefore not a product of nature. In this regard, in some embodiments, the phrase “derived from ginger” can be used interchangeably with the phrase “isolated from ginger” to describe a nanoparticle of the presently disclosed subject matter. For example, in some embodiments of the presently disclosed subject matter, nanoparticles derived from ginger can be produced by first grinding fresh ginger rhizome roots in a blender at high speeds and for a sufficient period of time to produce a juice of the ginger rhizome root. The ginger juice can then be subsequently and sequentially centrifuged at increasing speeds and for increasing periods of time (e.g., 1000 g for 10 min, 3000 g for 20 min, and 10,000 g for 40 min) to produce a microparticle pellet and supernatant. That resulting supernatant can then be further centrifuged at higher speeds and for an additional period of time (e.g., 150,000 g for 90 min) and subsequently exposed to a sucrose purification for isolation of nanoparticles using a sucrose step gradient (8% / 30% / 45% / 60%). Using such a sucrose step gradient, in some embodiments, different sub-populations of ginger-derived nanoparticles can be produced and obtained having various lipid compositions. For example, in some embodiments, by making use of the methods described herein, a sub-population of ginger-derived nanoparticles can be isolated from a band appearing between the 8% and 30% layers, and which have an average diameter of between 300 nm and 400 nm. In other embodiments, a sub-population of nanoparticles can be isolated from a band appearing between the 30% and 45% layer, and which have an average diameter of about 200 nm to about 300 nm. In some embodiments of the presently disclosed subject matter, the ginger-derived nanoparticles have an average diameter of about 100 nm to about 1000 nm and, in some embodiments, the ginger-derived nanoparticles produced by the presently-described methods are comprised of a phosphatidic acid (PA), a digalactosyldiacylglycerol (DGDG), a monogalactosyl monoacylglycerol (MGMG), and combinations thereof. For example, in some embodiments, the ginger-derived nanoparticles are comprised of about 30% to about 40% PA, about 30% to about 40% DGDG, and about 10% to about 20% MGMG. In some embodiments, the ginger-derived nanoparticle includes an effective amount of and/or are enriched with shogaol. For further information and guidance regarding the production of plant-derived nanoparticles, see, e.g., Ju, et al. Mol. Ther. 2013; 21(7): 1345-57, which is incorporated herein by reference in its entirety.

In some embodiments of the presently disclosed subject matter, a pharmaceutical composition is thus provided that comprises a ginger-derived nanoparticle disclosed herein and a pharmaceutical vehicle, carrier, or excipient. In some embodiments, the pharmaceutical composition is pharmaceutically acceptable in humans. Also, as described further below, in some embodiments, the pharmaceutical composition can be formulated as a therapeutic composition for delivery to a subject.

A pharmaceutical composition as described herein preferably comprises a composition that includes a pharmaceutical carrier such as aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. Additionally, the formulations can be presented in unit-dose or multi-dose containers, for example sealed ampoules and vials, and can be stored in a frozen or freeze-dried or room temperature (lyophilized) condition requiring only the addition of sterile liquid carrier immediately prior to use.

In some embodiments, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone, hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of capsules, tablets or lozenges formulated in a conventional manner.

Various liquid and powder formulations can also be prepared by conventional methods for inhalation into the lungs of the subject to be treated or for intranasal administration into the nose and sinus cavities of a subject to be treated . For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired compound and a suitable powder base such as lactose or starch.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compositions can be administered by the drip method, whereby a formulation including a pharmaceutical composition of the presently disclosed subject matter and a physiologically acceptable excipient is infused. Physiologically acceptable excipients can include, for example, 5% dextrose, 0.9% saline, Ringer’s solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the composition can be prepared and administered as a suspension in an aqueous base or a pharmaceutically acceptable oil base, such as an ester of a long chain fatty acid, (e.g., ethyl oleate).

In addition to the formulations described above, the ginger-derived nanoparticle compositions of the presently disclosed subject matter can also be formulated as rectal compositions, such as suppositories or retention enemas, e.g., containing conventional suppository bases such as cocoa butter or other glycerides. Further, the ginger-derived nanoparticle compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt capable of use in a therapeutic application.

Turning now to the therapeutic uses of the ginger-derived nanoparticles of the presently disclosed subject matter, in some embodiments, methods for treating alcohol-induced liver injury are provided. In some embodiments, a method for treating alcohol-induced liver injury is provided that comprises administering to a subject in need thereof an effective amount of a ginger-derived nanoparticle. In some embodiments, a method for treating alcohol-induced liver injury is further provided that includes a step of selecting a ginger-derived nanoparticle (e.g., a GDN having an average diameter of 300 nm to 400 nm) prior to administering the nanoparticle to a subject.

As used herein, the terms “treatment” or “treating” relate to any treatment of a condition of interest (e.g., an alcohol-induced liver injury), including but not limited to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

As used herein, the term “alcohol-induced liver injury” is used to refer to injury to a liver of a subject that is caused directly or indirectly by the consumption of alcohol (e.g., ethanol). Such alcohol-induced liver injury can be characterized by increased lipid droplets and fatty acids in the liver of a subject (e.g., a fatty liver), hepatitis, increased liver triglyceride levels, increased liver weight, hemorrhage, mononuclear cell infiltrates, inflammation, fibrosis, and the like.

For administration of a therapeutic composition as disclosed herein (e.g., a ginger-derived nanoparticle), conventional methods of extrapolating human dosage based on doses administered to a murine animal model can be carried out using the conversion factor for converting the mouse dosage to human dosage: Dose Human per kg = Dose Mouse per kg / 12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg × 37 kg/sq m=3700 mg/m2.

Suitable methods for administering a therapeutic composition in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments, the ginger-derived nanoparticles disclosed herein are administered orally.

Regardless of the route of administration, the compositions of the presently disclosed subject matter are typically administered in amount effective to achieve the desired response. As such, the term “effective amount” is used herein to refer to an amount of the therapeutic composition (e.g., a ginger-derived nanoparticle, and a pharmaceutically vehicle, carrier, or excipient) sufficient to produce a measurable biological response (e.g., a decrease in alcohol-induced liver injury). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman’s the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington’s Pharmaceutical Sciences, 15th ed. Mack Pub. Co., Easton, Pennsylvania; and Speight et al., (1997) Avery’s Drug Treatment: A Guide to the Properties, Choice, Therapeutic Use and Economic Value of Drugs in Disease Management, 4th ed. Adis International, Auckland/ Philadelphia; Duch et al., (1998) Toxicol. Lett. 100-101:255-263.

In some embodiments of the therapeutic methods described herein, administration of a ginger-derived nanoparticle increases a protective effect in the liver of a subject and/or reduces one or more symptoms of liver injury in a subject. For instance, in some embodiments, administering the ginger-derived nanoparticles increases an amount of nuclear factor erythroid-2 related factor (Nrf2) activation. In other embodiments, administering the ginger-derived nanoparticles reduces an amount of reactive oxygen species (ROS) in the liver of the subject, reduces an amount of triglycerides in the liver of the subject, decreases a total weight of the liver of the subject, and/or decreases an amount of lipid droplets in the liver of the subject..

Various methods known to those skilled in the art can be used to determine an increase or a reduction in the above-described factors and symptoms associated with an alcohol-induced liver injury in a subject. For example, in certain embodiments, activation of Nrf2 can be measured by identifying nuclear translocation of Nrf2 in the nuclear extracts of liver cells using techniques from as electromobility shift assays (EMSA) or immunoassays techniques with nuclear extracts isolated from cells. In other embodiments, the amounts of expression of known Nrf2-regulated genes in a subject can be determined by probing for mRNA of the Nrf2-regulted cells in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, CA). Because the probes to which the nucleic acid molecules of the sample are bound are known, the molecules in the sample can be identified. In this regard, DNA probes for one or more of the mRNAs encoded by the Nrf2-regulted genes can be immobilized on a substrate and provided for use in practicing a method in accordance with the presently disclosed subject matter.

With further regard to determining increases or reductions in the above-described factors and symptoms associated with an alcohol-induced liver injury in samples, and as further examples, chromatography, histology, mass spectrometry, and/or immunoassay devices and methods can also be used to measure the inflammatory cytokines or chemokines in samples, although other methods can also be used and are well known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety. Any suitable immunoassay can be utilized, for example, enzyme-linked immunoassays (ELISA), radioimmunoassays (RIAs), competitive binding assays, and the like. Specific immunological binding of the antibody to the inflammatory molecule can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

With still further regard to the various therapeutic methods described herein, although certain embodiments of the methods disclosed herein only call for a qualitative assessment (e.g., the presence or absence of Nrf2 in the nucleus of a cell), other embodiments of the methods call for a quantitative assessment (e.g., an amount of increase in the level of reactive oxygen species in a subject). Such quantitative assessments can be made, for example, using one of the above mentioned methods, as will be understood by those skilled in the art.

The skilled artisan will also understand that measuring an increase or a reduction in the amount of a certain feature (e.g., ROS levels) or an improvement in a certain feature (e.g., inflammation) in a subject is a statistical analysis. For example, a reduction in an amount of triglycerides in the liver of a subject can be compared to control level of triglycerides, and an amount of triglycerides of less than or equal to the control level can be indicative of a reduction in the amount of triglycerides, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

Still further provided, in some embodiments, are methods of decreasing nuclear factor erythroid-2 related factor (Nrf2) activation in a hepatocyte. In some embodiments, a method of decreasing nuclear factor erythroid-2 related factor (Nrf2) activation in a hepatocyte is provided that comprises contacting the hepatocyte with an effective amount of a ginger-derived nanoparticle. In some embodiments, the hepatocyte contacted with the ginger-derived nanoparticles is present within a subject.

As used herein, the term “subject” includes both human and animal subjects. Thus, veterinary therapeutic uses are provided in accordance with the presently disclosed subject matter. As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently disclosed subject matter is further illustrated by the following specific but non-limiting examples.

### EXAMPLES

Human daily exposure to nanoparticles from edible plants is inevitable, but significant advances are required to determine whether edible plant nanoparticles are beneficial to our health. Additionally, strategies are needed to elucidate the molecular mechanisms underlying any beneficial effects. In the following examples, a mouse model was used and showed that orally given nanoparticles isolated from ginger extracts using a sucrose gradient centrifugation procedure resulted in protecting mice against alcohol induced liver injury. The ginger derived nanoparticle (GDN)-mediated activation of Nrf2, led to the expression of a group of liver detoxifying/antioxidant genes and inhibited the production of reactive oxygen species (ROS), which partially contributed to the liver protection. Using lipid knock-out and knock-in strategies, the compound shogaol in the GDN was further identified as playing a role in the induction of Nrf2 in a TLR4/TRIF dependent manner. Given the critical role of Nrf2 in modulating numerous cellular processes, including hepatocyte homeostasis, drug metabolism, antioxidant defenses, and cell cycle progression of liver, this finding not only opened up a new avenue for investigating GDN as a means to protect against the development of liver related diseases such as alcohol induced liver injury or damage, but the finding also shed light on studying the cellular and molecular mechanisms underlying inter-species communication in the liver via edible plant derived nanoparticles.

### Material and Methods for Examples 1-4

Isolation and characterization of ginger-derived nanoparticles.. Fresh ginger rhizome roots were purchased from a local market, washed 3x with PBS. 200 g of washed roots were ground in a mixer (Osterizer 12-speed blender) at the highest speed for 10 min (pause 1 min for every 1 min blending). Ginger juice was then sequentially centrifuged at 1000 g for 10 min, 3000 g for 20 min and 10,000 g for 40 min. After 10,000 g centrifugation, the pellet was resuspended in PBS and referred to as microparticles. The supernatant was then centrifuged at 150,000 g for 90 min, the pellet was resuspended in PBS and transferred to a sucrose step gradient (8%/30%/45%/60%) and centrifuged at 150,000 g for 120 min. The bands between the 8%/30% layer and the 30%/45% layer were harvested separately and noted as GDN and ginger-derived exosome-like nanoparticles (GDEN2). The protein concentration of the samples was determined using a BCA assay kit (Thermo Scientific).

Mice. C57BL/6j mice, 6-8 weeks of age were obtained from Jackson Laboratories. MyD88, TRIF, and TLR4 knockout mice on a B6 background were kindly provided by Dr. Shizuo Akira (University of Osaka, Osaka, Japan). All animal procedures were approved by the University of Louisville Institutional Animal Care and Use Committee. For the mice alcoholic liver disease (ALD) model, 8-week-old male C57BL/6j mice were fed a liquid diet containing 5% ethanol for 13 days and on day 14 the mice were gavaged with a single dose of ethanol (5 g/kg body weight, 30% ethanol). The GDN treatment studies were conducted by gavage administering GDN (50 mg/mouse/day) or PBS as a control for 7 days prior to the ethanol diet and then continuously giving the GDN or PBS to the mice after they were fed the 5% ethanol diet. On day 14, after the 30% ethanol feeding, and 9 hr post gavaged with the last GDN treatment, the mice were euthanized, serum and liver were harvested for examination.

Lipid extraction, thin layer chromatography (TLC) and lipidomic analysis. Total lipid extraction of GDENs was performed according to the method of Bligh and Dyer, and the lipids were dissolved in chloroform for analysis. The lipid composition was analyzed on a triple quadrupole tandem mass spectrometer (API 4000, Applied Biosystems, CA) as previously described. The data were reported as percentage of total signal of the molecular species after normalization of the signals to internal standards of the same lipid class.

For TLC analysis, lipids extracted as described above and stranded 6-Shogaol (Sigma-Aldrich, 10pMol) and 6-Gignerol (Sigma-Aldrich, 10pMol) were applied on a Silic gel 60A TLC plate (Whatman) and developed in a mixture of hexane:ethylacetate:formaic acid=55:40:5. For analysis of lipids extracted from ginger, ginger micro-particles, and GDN, TLC was developed with a mixture of toluene-ethyl acetate (3:1, v/v). Developed plates were initially air-dried, then sprayed with CuSCU-phosphoric acid reagent (10% CuSO₄ in 8% phosphoric acid), and followed by charring at 100° C. for 10 min.

To knock out Shogaol from GDN lipids, duplicated GDN derived lipid samples were loaded on the same TLC plate. A standard control of Shogaol (Sigma) was loaded next to GDN lipid samples and used to determine the position of Shogaol in the GDN lipids loaded on the same TLC plate. After separation on the TLC plate, one of the duplicate GDN derived lipid samples and the Shogaol standard were developed as a reference for the location of GDN Shogaol on the TLC plate. The band that had migrated to the same position as the standard Shogaol was removed for HPLC analysis and the rest of the fractions of GDN lipids in the TLC were collected and extracted with 2 mL of chloroform methanol (1:1, v/v) and 0.9 mL water. The organic phase samples were aliquoted and dried by heat under nitrogen (0.2 psi). Total lipids were determined using the phosphate assay as described. For assembling liposome-like nanoparticles (LN), the dried lipids were immediately suspended in distilled water (150-200 µl). After bath-sonication (FS60 bath sonicator, Fisher Scitific, Pittsburg, PA) for 5 min, an equal volume of buffer (308 mM NaCl, 40 mM Hepes, pH7.4) was added and sonicated for another 5 min. The charges and sizes of liposome-like nanoparticles were examined using a method as decribed previously.

Particle size and surface charge analysis. The particle size and zeta potential were measured by using Zetasizer Nano S90 as previously described.

Atomic force microscope (AFM). Specimens were prepared for Atomic Force Microscopy (AFM) using a conventional procedure. In brief, the GDN was fixed with 2% glutaraldehyde in 0.1 M cacodylate buffer (pH7.4) for 4 h, at 4° C. After extensively washing with 0.1 M cacodylate buffer (pH7.4), samples were fixed with 1% osmium tetroxide in 0.1 M cacodylate buffer (pH 7.4) for 1 h at 4° C., dehydrated in a graded ethanol (25% for 20 min, 50% for 20 min, 75% for 20 min, 95% for 20 min, 100% for 30 min, and 100% for 30 min). The samples were then examined using a MFP-3D™ AFM scope (Oxford Instrument).

In vitro digestion of GDN. In vitro digestion conditions were based on a previous description. In brief, 1 ml of GDN in a water solution were incubated with slow rotation at 37° C. for 60 min after the addition of 1.34 µl of 18.5% w/v HCl and 24 µl of a pepsin solution (80 mg/ml in 0.1 N of HCl, pH 2.0, Sigma) to form a stomach-like solution. Then, 80 µl of a mixture containing 24 mg/ml of bile extract and 4 mg/ml of pancreatin (Sigma) in 0.1 M of NaHCO₃ was added to the stomach-like solution. The pH value of the bulk solution was adjusted to 6.5 with 1 M NaHCO₃, which was referred to as an intestinal solution. GDN was incubated for an additional 60 min in the intestinal solution. The stability of GDN was evaluated by measuring particle size and surface charge.

High-performance liquid chromatography (HPLC) analysis for Shogaol. Lipid extracts from GDN or banded lipids from TLC plate were dried under nitrogen gas and dissolved in methanol. Chromatography was performed on an Agilent 1120 system using an Eclipse plus C18 column. The mobile phase consisted of 20 mM HCl (A)/Acetonitrile (B). An aliquot (100 µL, sample or Shogaol) was injected and eluted with reagent B (25%) for 6 min, then with a continuous gradient of reagent B from 25% to 100% in 24 min, reagent B (100%) for 2 min, and finally reagent B (25%) for 5 min. The UV detector was set at 283 nm. The analyses were performed at 25° C. with a 1 ml/min flow rate.

GDN labeling. For DiR labeling, GDNs (50 mg in 1 ml PBS) were mixed with 1 µL near-infrared lipophilic carbocyanine dye (1,1′-dioctadecyl-3,3,3′3′-tetramethyl-indotricarbocyanine-iodide, DiR, Invitrogen, Carlsbad, CA, 5 mM in DMSO) and incubated at 22° C. for 20 min. For PKH67 labeling, GDNs (50 mg in 1 ml Dilute C) were mixed with 2 µL PKH67 (Sigma, 1 mM in ethanol) and incubated at 37° C. for 5 min. Labeling was stopped by adding 1 mL exosomes depleted FBS (supernatant of centrifuging at 150,000 g for overnight). For IRDye-700DX labeling, GDN (5 mg in 1 ml PBS) were mixed with 1 µL IRDye-700DX NHS Ester (5 mg/ml in DMSO) and incubated at 37° C. for 2 hr. All unlabeled dye was washed away by centrifugation at 150,000 g for 90 min, labeled GDN pellets were resuspended in PBS.

Biodistribution and cellular targeting of orally administrated GDN. After orally given 50 mg of either DiR or PKH26 fluorescent dye (Sigma) labeled GDN, mice were sacrificed at different time points and small intestine, colon, mesenteric lymph node (MLN), spleen and liver tissues were used for imaging. DiR fluorescent signal was detected and measured using the Imaging Station 4000 mm PRO (Kodak Carestream) and quantified using the Carestream MI software. PKH26 signal in frozen tissue sections was observed using the Nikon AIR Confocal system.

For tracing GDN traffic in blood, IRDye-700DX and PKH67 double labeled GDN (200 µg) were orally administrated into naive mice or mice fed with liquid alcoholic diet (6 hr after final binge). Plasma samples were collected at different time points. Each of the plasma samples (40 µL) collected was mixed with 60 µL PBS, loaded into 96-wells plate and scanned by Li-CoR Scanner. The amount of GDN in plasma was calculated based on the standard curve made from IRDye-700DX labeled GDN. A standard curve was created by plotting the mean absorbance for each IRDye-700DX labeled GDN standard diluted in naive mouse plasma against the GDN protein concentration. The amount of GDN in plasma after intravenous injection of IRDye-700DX labeled GDN (10 µg) was determined using the same method as described above for gavage-administration.

To further validate the data generated from the protocol described above, mice were gavage-given IRDye-700DX and DiR double labeled GDN (200 µg) or PBS as a control. Forty-five min after the oral administration, plasma was collected and diluted in PBS at 1:10 and pelleted by ultracentrifugation at 150,000 g for 2 hr. The pellets were scanned for measuring the intensity of the fluorescence signal of IRDye-700DX and DiR using the Li-CoR. The fold changes of fluorescent intensity was expressed as fluorescent intensity of the pellets purified from GDN treated mice /PBS treated mice.

AST and ALT measurement. To test for hepatotoxicity, levels of ALT and AST activity in serum were measured using the Infinity Enzymatic Assay Reagent (Thermo Scientific).

H&E staining and immunofluorescence staining. For histopathology, H&E staining was performed on paraffin-embedded liver sections. For immunofluorescence analysis, intestinal tissues or liver tissues were fixed in cold 2% paraformaldehyde solution for 2 hr at 4° C. Fixed tissues were dehydrated in graded sucrose solution in PBS (5% for 2 hr, 10% for 2 hr and 20% overnight) at 4° C. OCT (TissueTek)-embedded tissue were frozen fixed at -80° C. Slides were hydrated in PBS and stained with a rat monoclonal anti-CD31 (390, eBioscience), a goat polyclonal anti-Lyve-1 (R&D system), a mouse anti-albumin antibody (R&D system), or a rat anti-mouse F4/80 (Biolegend). After washing, cells were stained with a Alexa Fluor 488 labeled rabbit anti-mouse, a goat anti-rat, or a donkey anti-goat antibody (Invitrogen Life Sciences). For staining with Nrf2 antibody, primary hepatocytes fixed in cold 4% paraformaldehyde for 20 min were permeabilized with 1% Triton-X 100 in PBS for 2 min on ice, followed by blocking with 5% BSA in PBS containing 0.1% Triton-X 100 for 1 h. The hepatocytes were then stained with a rabbit anti-mouse Nrf2 polyclonal antibody (Santa Cruz Biotechnology) for 2 hr at 22° C. After washing, cells were stained with a Alexa Fluor 488 labeled goat anti-rabbit antibody (Invitrogen Life Sciences). Slides were stained by DAPI (4,6-diamidino-2-phenylindole; S36938; Molecular Probes and Invitrogen Life Sciences) for 90s and mounted using fluorogel with Tris buffer (Electron Microscopy Science). The stained hepatocytes or the stained sliced liver or intestinal tissues were assessed using a Nikon AIR Confocal system. Fold changes in Nrf2 nuclear translocation between treatments and controls were determined by the following method. To quantify the nuclear to cytoplasmic ratios of Nrf2 distribution, the DAPI image of nuclei was used and a mask was applied to segment the Nrf2 image to obtain nuclear Nrf2 content. Then, intensity of Nrf2 florescent imaging from the cell minus its nuclear Nrf2 content was taken as the cytoplasmic Nrf2. The ratios were then obtained for each nucleus-cytoplasm pair of a hepatocyte. Using a confocal microscope a total of 5 fields for each treatment were analyzed. Mean ratios of each treatment were then calculated using the following formula: Mean ratios = the sum of ratios obtained from Nrf2 florescent intensity of nucleus/cytoplasm of each cell divided by the total numbers of cells analyzed. Data were represented as mean fold change obtained from comparing GDENs or LN treated to PBS-treated hepatocytes. Data are mean ± SEM (n = 5). *P < 0.05, **P < 0.01.

Primary hepatocyte isolation, culture and uptake of GDENs. Hepatocytes were isolated from 8-week-old adult C57/B6 and TLR4, Trif, Myd88 knockout mice using a two-step collagenase perfusion procedure. Each liver was perfused via portal vein with 30 mL 37° C. pre-warmed perfusion buffer (HBSS without Ca²⁺ and Mg²⁺ (Thermo Scientific), containing 0.2 mM EDTA and 20 mM glucose) and followed by 30 mL 37° C. pre-warmed digestion buffer (HBSS with Ca²⁺ and Mg²⁺ (Thermo Scientific) containing 20 mM glucose and 100 U/L collagenase type I (Worthington-Biochem). Digested livers were transferred to a chilled dish and dissociated cells were isolated by gentle teasing apart of the liver with 1 mL pipette tips. Hepatocytes were washed twice with DMEM/D-12 (1:1) (Thermo Scientific) media at 100 g for 4 min at 4° C.The washed hepatocytes were purified on a 40%/90% Percoll gradient centrifuged at 700 g for 20 min at 20° C. Hepatocytes were plated at a density of 3.5_{*}10⁴/well (96-well plate) or 1.5×10⁵/well (24-well plate) in cell culture plates pre-coated with collagen (Type I from rat nail, BD Biosciences, 50 µg in 1 mL 30% ethanol, dried under air for more than 12 hr) and incubated in 5% CO₂ at 37° C.

To study the effect of endocytosis inhibitors [(Amiloride (50 µM), Bafilomycin Al (10 nM), Chlorpromazine (5 pM), Cytochalasin D (1 µM), Imdomusine (50 µM) or Nocodazole (25 µM)] (all of them purchased from Sigma-Aldrich) on GDN and GDEN2 uptake, hepatocytes were cultured at 37° C. in the presence of an endocytosis inhibitor for 1 h prior to the addition of PKH26-labeled GDN or GDEN2 for an additional 3 h culture period. After washing with PBS 3x, cells were fixed in a cold 4% paraformaldehyde solution for 20 min, and blocked with 5% BSA in PBS. Cells were then stained with a mouse anti-albumin antibody (R&D system) for 1 h at 22° C. After washing the cells were stained with a Alexa Fluor 488 labeled rabbit anti-mouse antibody (Invitrogen Life Sciences). The cells were washed and counterstained with DAPI and images were captured using a Nikon AIR confocal microscope equipped with a digital image analysis system (Pixera).

To determine the effects of temperature on GDN uptake, primary hepatocytes were isolated and cultured overnight at 37° C. in a CO₂ incubator. The next day, cultured hepatocytes were treated with PKH26-labelled GDN and continued in culture at 37° C., 20° C. or 4° C. for an additional 6 h. Then, the cells were washed in PBS and stained with anti-albumin antibody and imaged with a confocal microscopy.

Quantification of ROS production. Hepatocytes in 96-well plates were cultured for 24 hr in the presence of GDN or GDEN2 (100 µg/ml) or liposomes assembled from GDN or GDEN2, and then stimulated by adding ethanol (150 mM). 12 hr after stimulation the culture media was replaced with Carboxyl-H₂DCFDA (5 µM in PBS) (Molecular Probes) and continued in culture for 30 min at 37° C., in a 5% CO₂ incubator. Unincorporated dye was removed by washing with PBS for 2x. Accumulation of DCF in hepatocytes was measured by an increase in fluorescence of ROS oxidation product, DCF. DCF can be measured at a 485/20 nm excitation and a 528/20 nm emission with a Synergy HT Multi-Mode Microplate Reader (BioTek) or analyzed with a flow cytometer (BD FACSCalibur; BD Biosciences) after hepatocyte trypsinization. Mean DCF fluorescence intensity was calculated based on measurements of 20,000 cells using the FL1-H channel.

Cytoplasmic and nuclear protein extraction. To prepare nuclear protein extracts, hepatocytes were washed with a cold perfusion buffer (HBSS without Ca²⁺ and Mg²⁺ (Thermo Scientific), containing 0.2 mM EDTA and 20 mM glucose), harvested by adding a digestion buffer (HBSS with Ca²⁺ and Mg²⁺ containing 20 mM glucose and 100 U/L collagenase type I (Worthington-biochem) followed by gentle scraping. After washing with cold PBS at 100 g for 4 min, the cell pellets were resuspended in cold cytoplasmic extract buffer (10 mM HEPES, 60 mM KC1, 1 mM EDTA, 1 mM DTT and 1 mM PMSF, pH 7.6) containing 0.075% (v/v) NP40. After incubated on ice for 3 min, the cell suspension was centrifuged at 400 g for 4 min, the supernatant (cytoplasmic protein) was collected and the pellet was washed with cytoplasmic extract buffer without NP40 one more time. Nuclear protein was extracted from the pellet with nuclear extract buffer (20 mM Tris Cl, 420 mM NaCl, 1.5 mM MgCl2, 0.2 mM EDTA, 1 mM PMSF and 25% (v/v) glycerol, pH 8.0). The proteins were quantified using a method as described previously.

Oil red O staining. Cryosections of liver were air-dried, rinsed with distilled water, stained with Oil Red O (Sigma-Aldrich) in 60% Isopropanol for 15 min and counterstained with hematoxylin for 10 min. An Aperio Sanscoper system was used to capture and analyze stained sections.

Hepatic triglyceride. Triglyceride in liver was measured according to the manufacturer’s instruction using the Triglyceride assay kit from Cayman.

RNA extraction and PCR. Total RNA was isolated from liver tissue with Trizol agent according to the manufacturer’s instructions (Invitrogen). Total RNA (1 µg) was reverse-transcribed with Superscript III and random primers (Invitrogen). For quantitation of genes interest, cDNA samples were amplified in a CFX96 Realtime System Bio-Rad Laboratories using a method as described previously. Fold changes in mRNA expression between treatments and controls were determined by the δCT method as described. Differences between groups were normalized to a GAPDH reference. All primers were purchased from Eurofins MWG Operon. The primer pairs for analysis are provided in Table 1.

TABLE 1

Primers used for Real-Time PCR

| | |
|---|---|
| Gene | Primers |
| GCLC | Forward: 5′-ACATCTACCACGCAGTCAAGGACC-3′ (SEQ ID NO: 1) Reverse: 5′-CTCAAGAACATCGCCTCCATTCAG-3′ (SEQ ID NO: 2) |
| GCLM | Forward: 5′-GCCCGCTCGCCATCTCTC-3′ (SEQ ID NO: 3) Reverse: 5′-GTTGAGCAGGTTCCCGGTCT-3′ (SEQ ID NO: 4) |
| NQO1 | Forward: 5′-AGCGTTCGGTATTACGATCC-3′ (SEQ ID NO: 5) Reverse: 5′-AGTACAATCAGGGCTCTTCTCG-3′ (SEQ ID NO: 6) |
| HO-1 | Forward: 5′-ACGCATATACCCGCTACCTG-3′ (SEQ ID NO: 7) Reverse: 5′-CCAGAGTGTTCATTCGAGCA-3′ (SEQ ID NO: 8) |

Western Blot Analysis. Western blots were carried out as described previously. In brief, proteins were separated on 10% polyacrylamide gels using SDS-PAGE and transferred to nitrocellulose membranes. Membranes were probed with specific antibodies: rabbit anti-mouse Nrf2 polyclonal antibody (Santa Cruz Biotechnology), rabbit monoclonal anti-GAPDH antibody (D16H11, from Cell Signaling Technology) or mouse monoclonal anti-PCNA antibody (PC10, from Santa Cruz). After washing, membrane was stained by Alexa fluor 680 labeled secondary antibody and signal intensity was quantified with an Odyssey instrument (Li-CoR Bioscience, Lincoln, NE) and a previously described protocol.

Statistical analysis. One-way, two-way analysis of variance (ANOVA) and t-test were used to determine statistical significance. (*p<0.05, and **p<0.01).

### Example 1 - Characterization of Ginger Derived Nanoparticles (GDN)

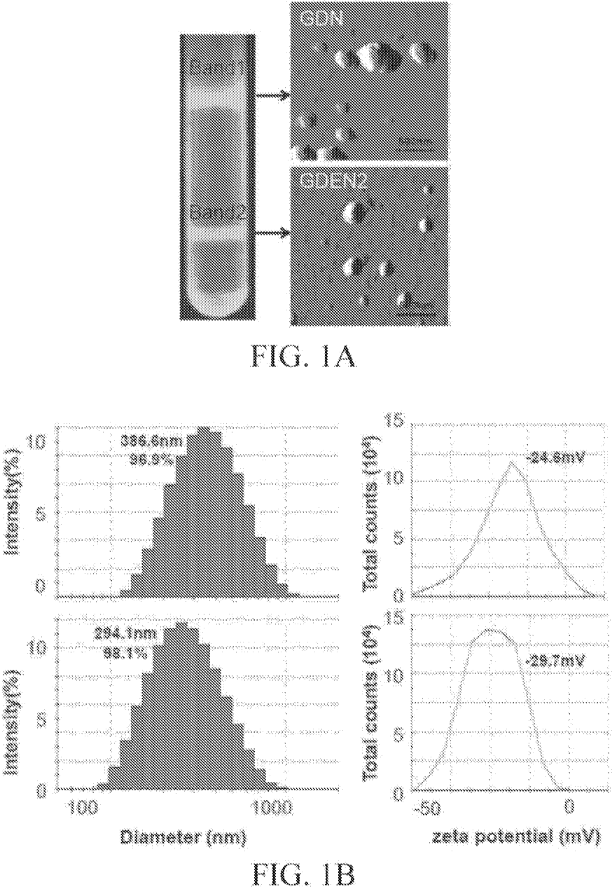

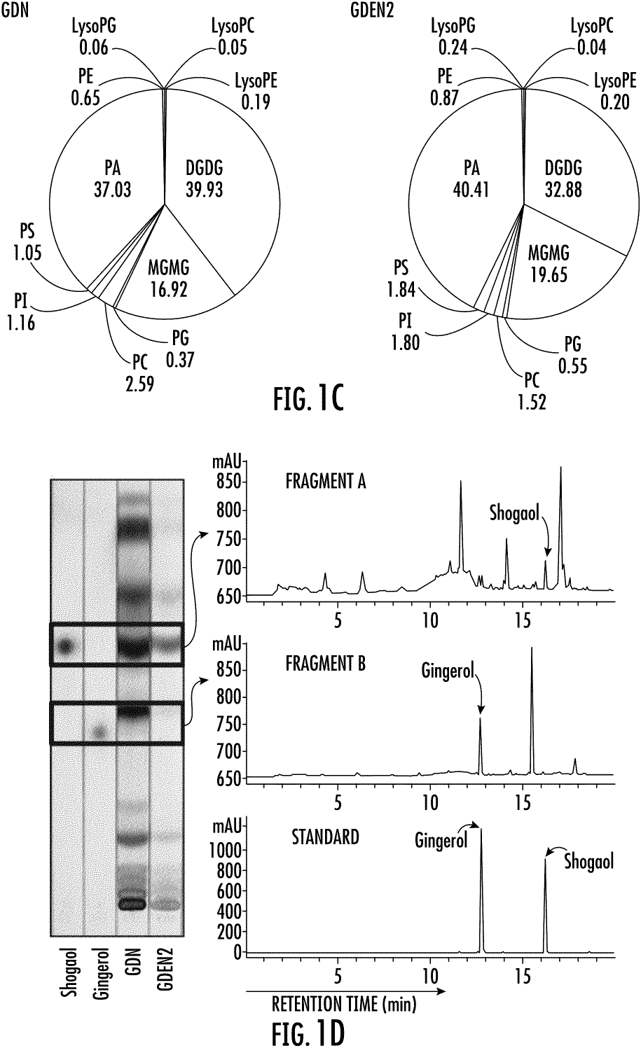

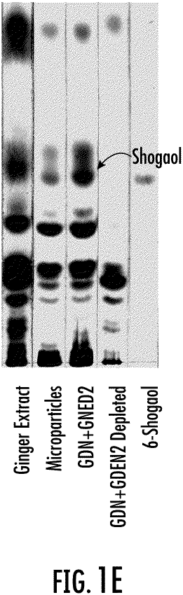

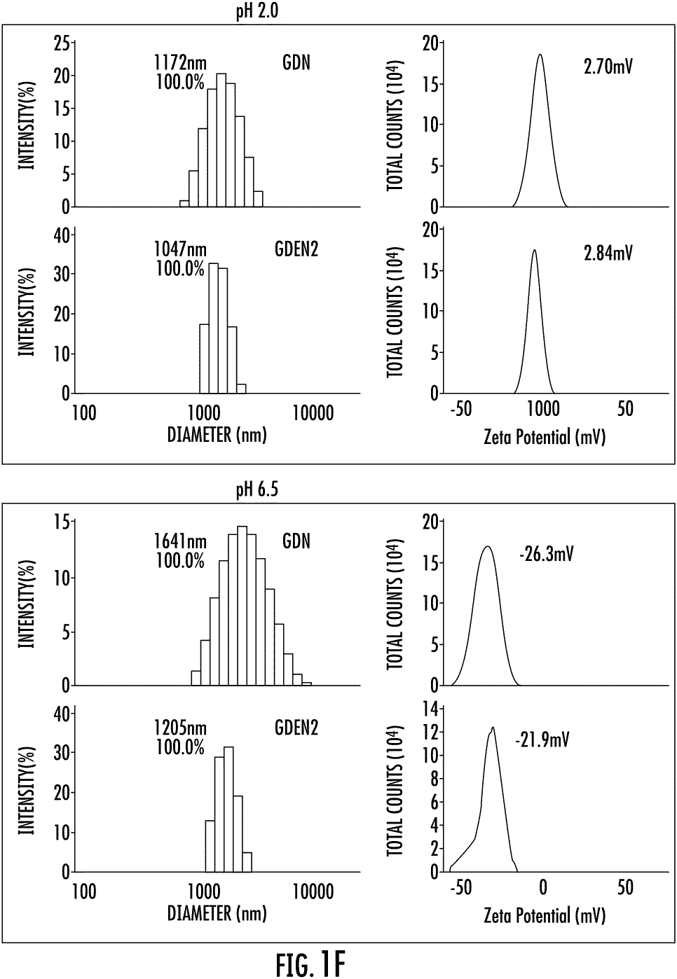

Ginger derived nanoparticles were isolated from homogenized ginger using a sucrose gradient centrifugation method. The majority of the ginger derived nanoparticles accumulated at the 8%/30% interface (band 1, referred it to as GDN, 3.79 ±0.27 mg/g of ginger protein), and at the 30%/45% interface (band 2, 0.31±0.01 mg/g of ginger protein) of the sucrose gradient. Band 2 has been characterized in a manuscript published previously and the particles in that band were referred to as ginger derived exosome-like nanoparticles (GDEN2) and used as a reference. GDN and GDEN2 integrity and size were evaluated by atomic force microscopy (AFM) (FIG. 1A) and a nano zetasizer (FIG. 1B). The results showed that the size distribution of the nanoparticles of isolated GDN ranged from 102.3 to 998.3 nm in diameter, with an average diameter of 386.6 nm (GDN), and an average diameter of 294.1 nm for the GDEN2 population. Zeta potential measurements indicated that ginger nanoparticles had a negative zeta potential value ranging from -24.6 mV (GDN) to -29.7 mV (GDEN2). Lipidomic data (FIG. 1C) indicated that both GDN and GDEN2 were enriched with phosphatidic acids (PA) (37.03 and 40.41%, respectively), digalactosyldiacylglycerol (DGDG) (39.93, 32.88%, respectively), and monogalactosyl monoacylglycerol (MGMG) (16.92, 19.65%, respectively). Interestingly, among the lipids analyzed, shogaols were much higher in GDN than in GDEN2 (FIG. 1D), even though total lipids extracted from equal amount of ginger nanoparticles used were loaded on the TLC plate, the other lipids, as indicated in Table 2, were also much higher in GDN than in GDEN2. TLC analysis further indicate that most of the shogaols in the ginger extracts are not present in a free form but are associated with either ginger nanoparticles or microparticles isolated from ginger extracts (FIG. 1e) as indicated by the fact that depletion of GDN and GDEN2 from ginger extracts led to no visible shogaol on the developed TLC plate.

TABLE 2

Lipids in GEDNs (ng/rng of dry GEDNs)

| Lipid | GDN | GDEN2 |
|---|---|---|
| DGDG | 0.353 | 0.084 |
| MGMG | 0.149 | 0.050 |
| PG | 0.003 | 0.001 |
| PC | 0.023 | 0.004 |
| PI | 0.010 | 0.005 |
| PS | 0.009 | 0.005 |
| PA | 0.327 | 0.103 |
| PE | 0.006 | 0.002 |
| LysoPG | 0.001 | 0.001 |
| LysoPC | 0.000 | 0.000 |
| LysoPE | 0.002 | 0.001 |
| Total polar lipid | 0.883 | 0.256 |

DGDG; Digalactosyldiacylglycerol
MGMG: Monogalactosyl Monoacylglycerol
PG: Phosphatidylglycerol
PC: Phosphatidylcholine
PI: Phosphatidylinositol
PS: Phosphatidylserine
PA: Phosphatidic acids
PE: Phosphatidylethanolamine
LysoPG: Lysophosphatidylglycerol
LysoPC: Lysophosphatidylcholine
LysoPE: Lysophosphatidylethanolamine

To test the stability of ginger nanoparticles under physiological conditions, in vivo conditions were mimicked by suspending ginger nanoparticles in a stomach-like solution (pH 2.0) or a small intestinal-like solution (pH 6.5). Interestingly, the results showed that compared to the size of ginger nanoparticles in PBS (FIG. 1B), the diameter of ginger nanoparticles was increased in a stomach-like solution, and were further enlarged a small intestine-like solution. Moreover, the ginger nanoparticles surface charge in a stomach-like solution changed from negative to a positive charge; whereas, in a small intestine-like solution, the ginger nanoparticles shifted back from a positive to a negative charged surface (FIG. 1F).

### Example 2 - In Vivo Distribution of Orally Administered Ginger Nanoparticles

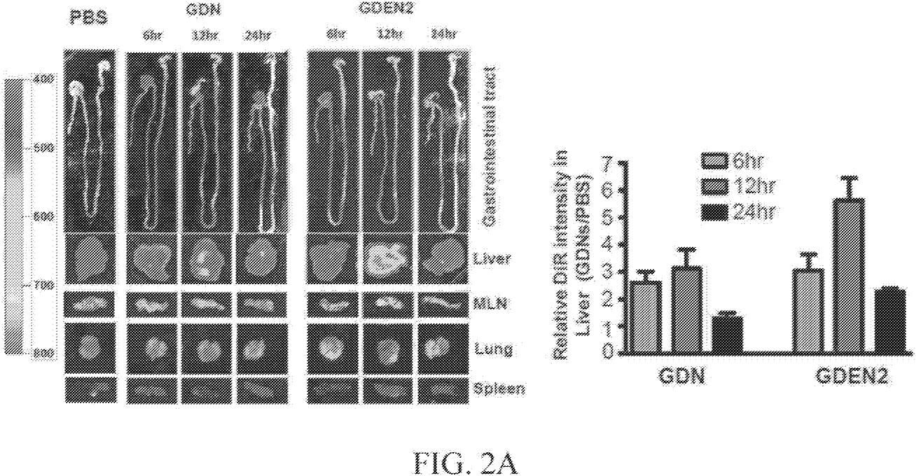

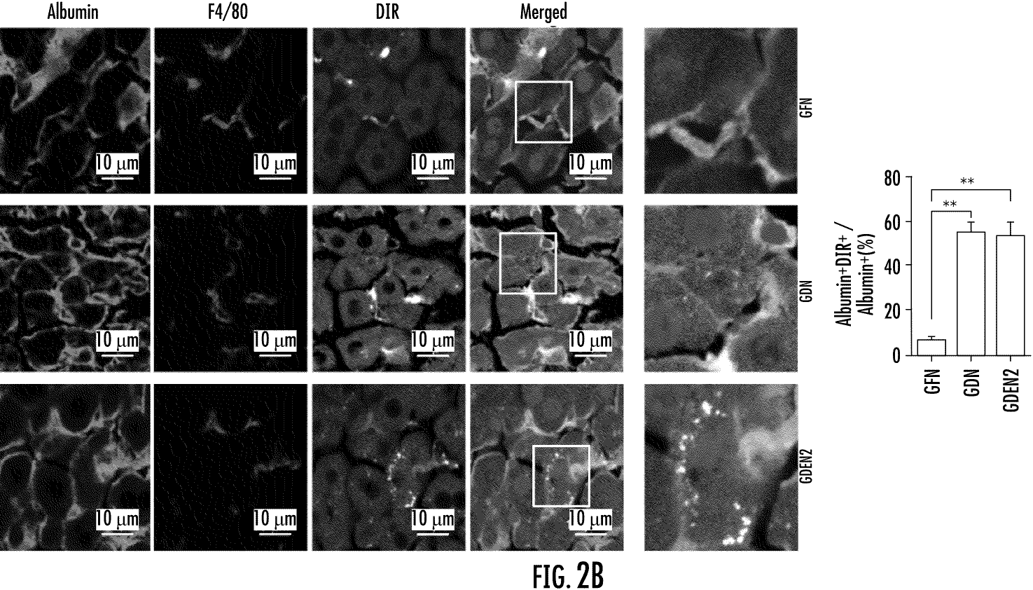

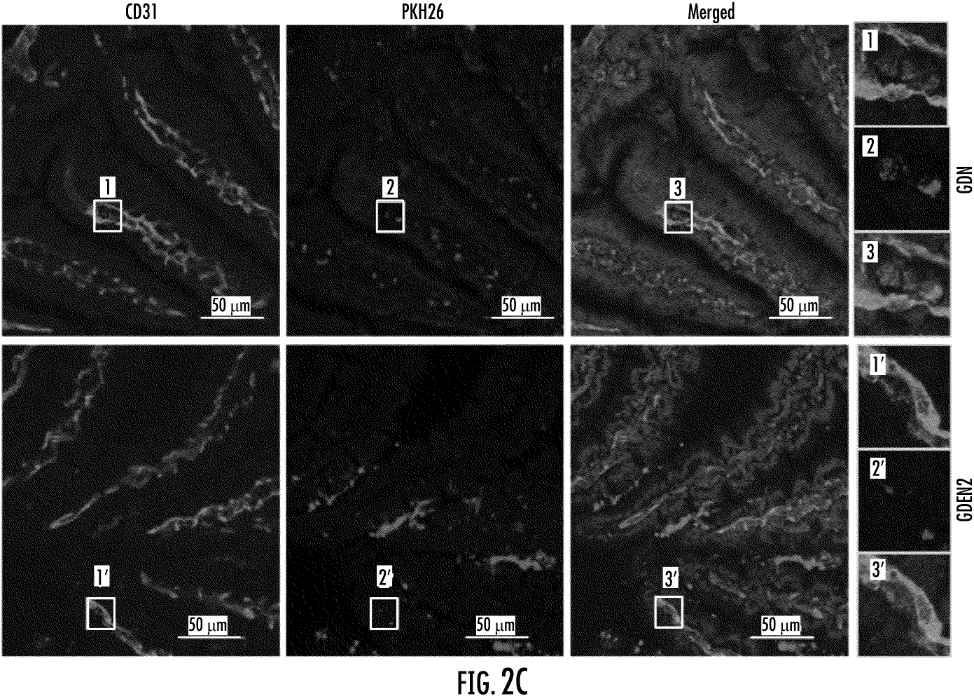

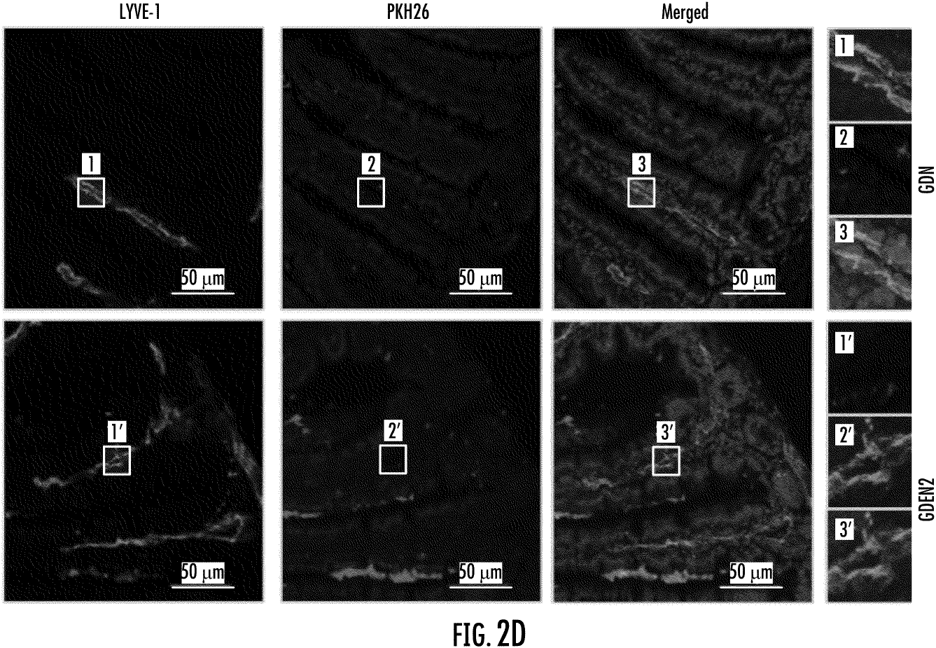

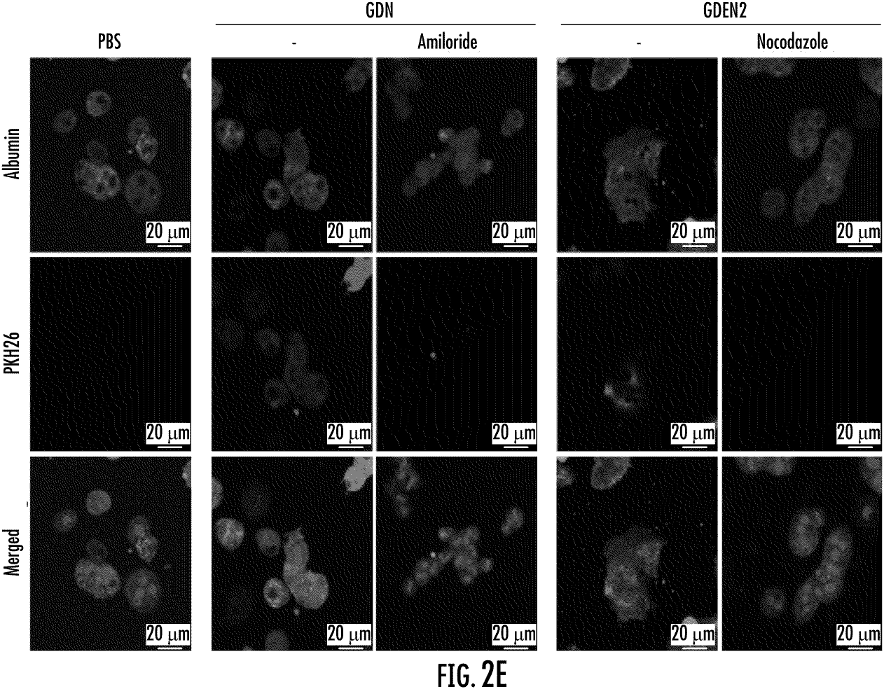

To determine the tissue distribution of ginger nanoparticles, in vivo biodistribution of DiR-labeled ginger nanoparticles was evaluated in mice using a Kodak Image Station 4000 MM Pro system. After oral administration, DiR fluorescent signals were predominantly detected in liver with a peak intensity at 12 hrs, and in mesenteric lymph nodes (MLN); however, fluorescent signals were not detected in the lung, spleen (FIG. 2A) or other organs. The presence of DiR labeled ginger nanoparticles in the liver was further confirmed by confocal immune staining for albumin (FIG. 2B), indicating that hepatocytes are the primary cells targeted by ginger nanoparticles. Albumin⁺ hepatocytes are ginger nanoparticles specific; whereas, most of nanoparticles from grapefruit are co-localized with F4/80⁺ liver Kupffer cells but not albumin⁺ hepatocytes (FIG. 2B, top panels). The co-localization of the PKH26 signals with CD31, a marker of endothelial cells (FIG. 2C), but not LYVE1, a marker of lymphatic capillaries (FIG. 2D), along the length of the intestinal endothelial vessels within 6 h of administration of ginger nanoparticles suggests that the ginger nanoparticles migrate into the liver from the gut primarily through vascular vessels. Thus, ginger nanoparticles can gain access and traffic within the vascular system of the liver.

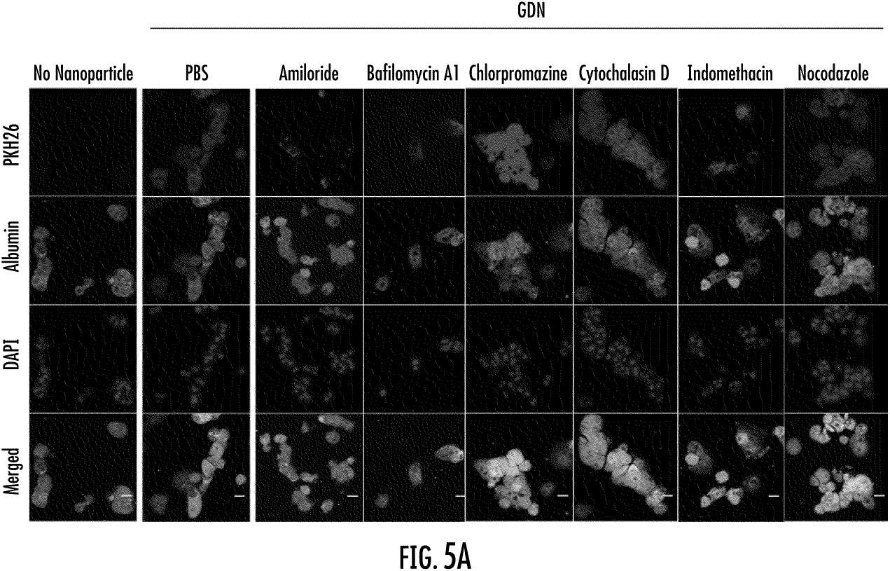

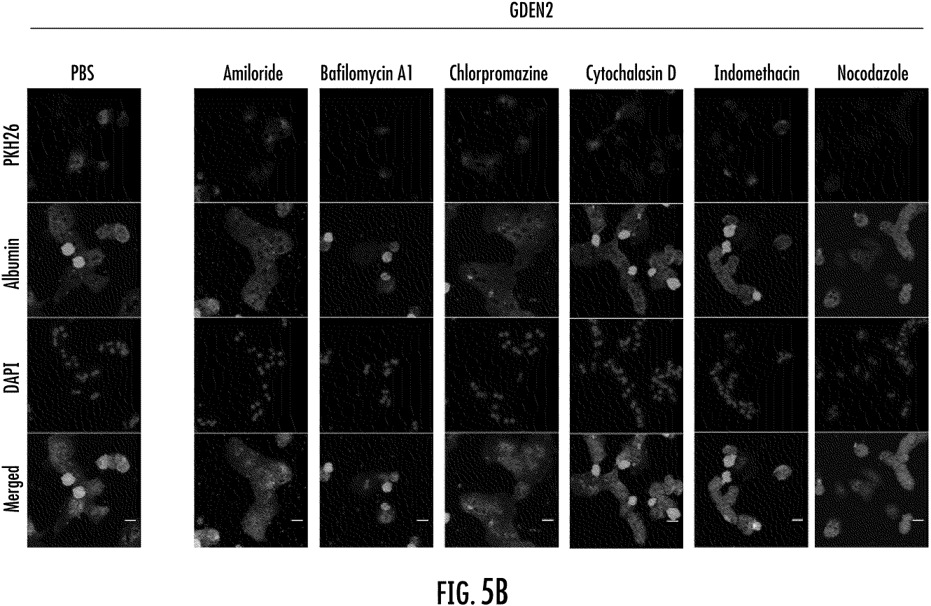

To further examine the mechanism of GDNs internalization, primary hepatocytes were treated with endocytosis inhibitors. Uptake of PKH26-GDN (FIG. 2E) was markedly inhibited by amiloride, an inhibitor of macropinocytosis, and uptake of PKH26-GDEN2 was inhibited by the nocodazole, an inhibitor of the polymerization of microtubules. Uptake of PKH26-GDN and PKH26-GDEN2 was not greatly diminished by treatment of primary hepatocytes with other inhibitors as listed (FIGS. 5A-5B), suggesting specificity of endocytosis pathways of GDN and GDEN2.

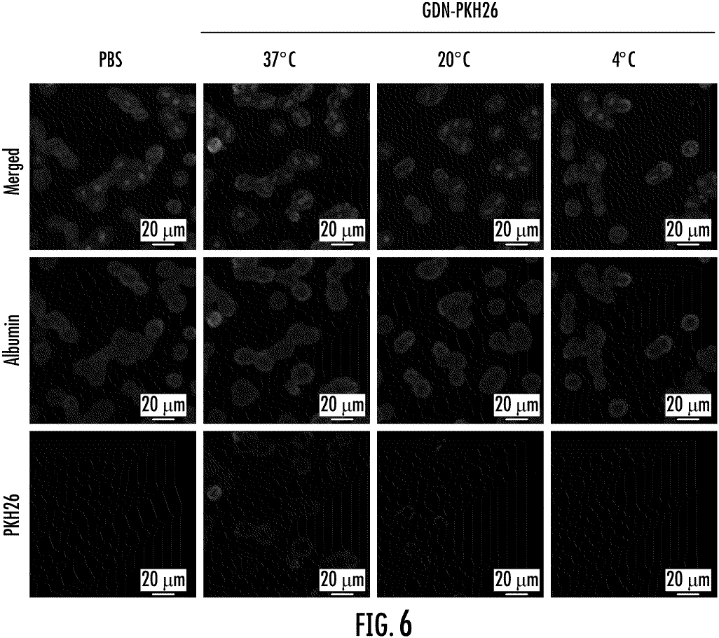

The efficiency of uptake of GDN was further demonstrated as a temperature-dependent process. Uptake rates were very slow at 4° C. and increased as the temperature was raised (FIG. 6), suggesting that metabolic energy is required for this process.

### Example 3 - Activation of Nuclear Factor Erythroid-2-Related Factor-2 (Nrf2) Is Dependent on GDN 6-Shogaol Through TLR4/TRIF Pathway

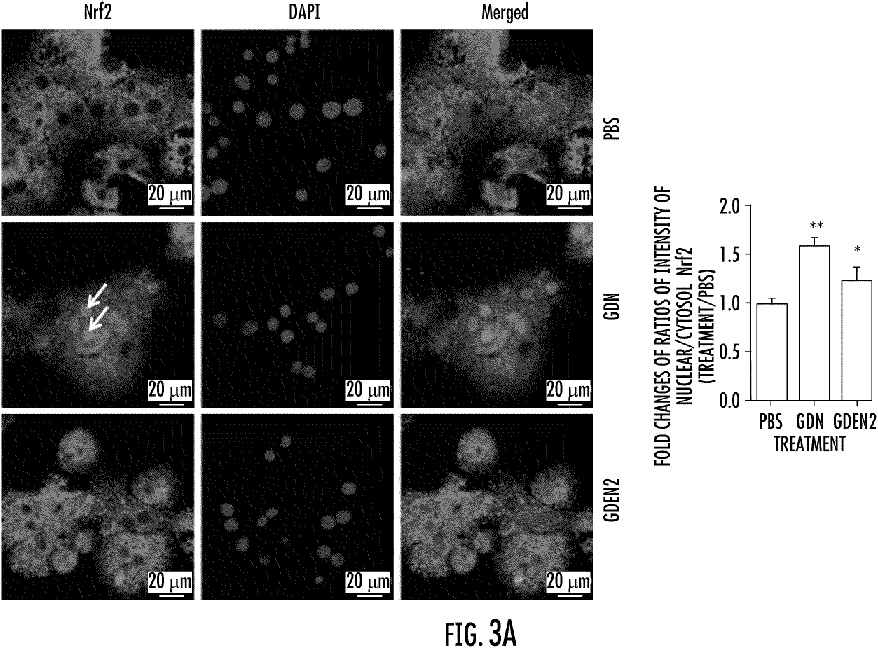

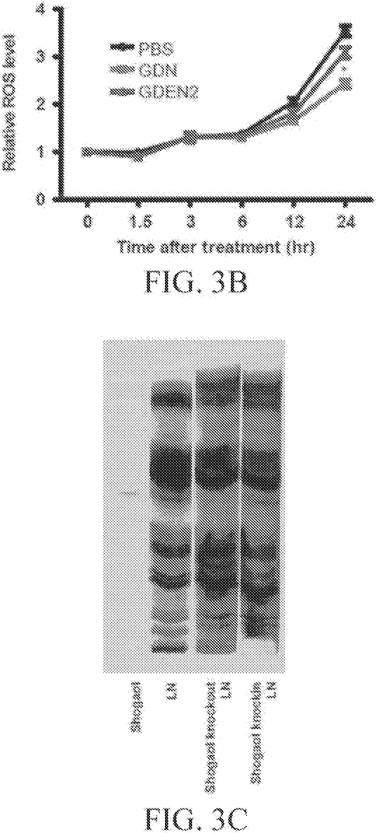

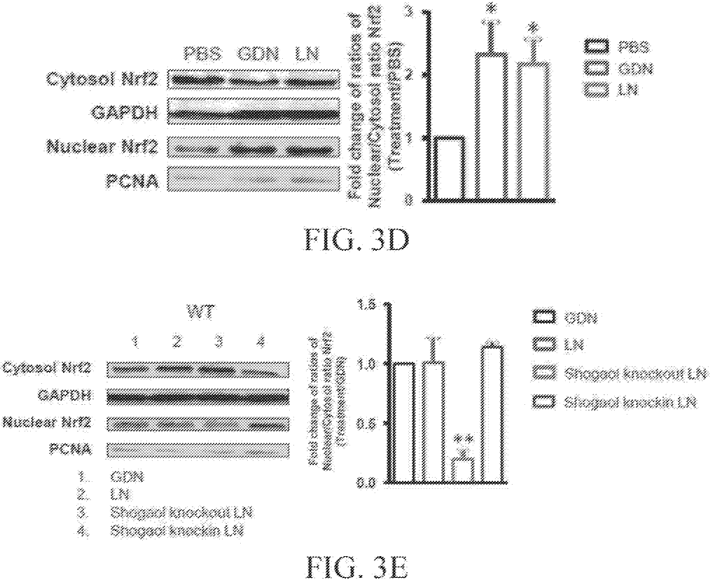

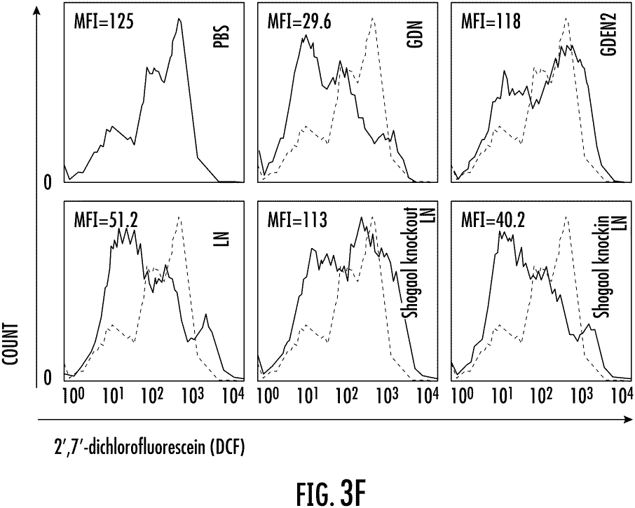

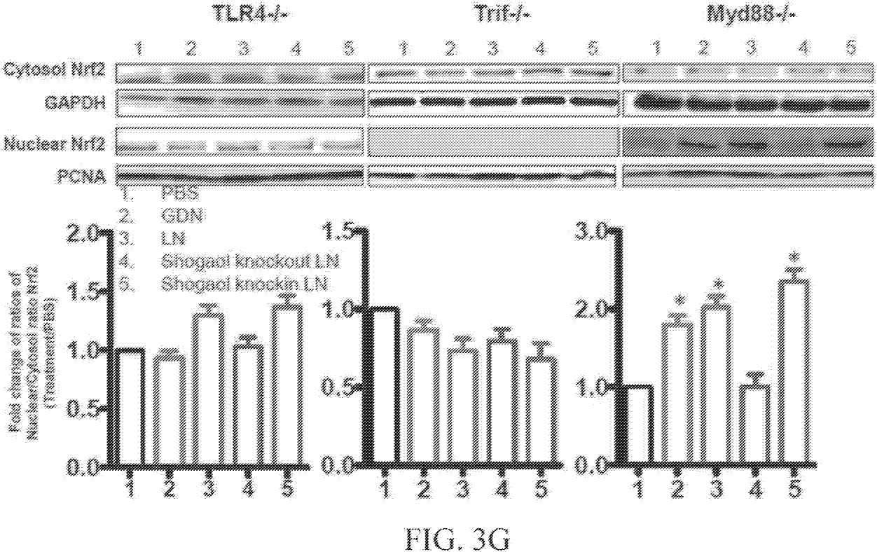

The above-described data indicated that shogaol content in GDN was higher than in GDEN2 (FIG. 1D). A shogaol-rich ginger extract may enhance antioxidant defense mechanisms through the induction of Nrf2. To determine whether shogaol-rich GDN have a different effect on Nrf2 activation when compared to GDEN2, primary hepatocytes were treated with GDENs. The nuclear translocation of Nrf2 was analyzed. The results from immune-staining of Nrf2 (FIG. 3A) showed that primary hepatocytes treated with GDN have a significantly increased nuclear translocation of Nrf2 when compared to cells treated with PBS. This result was also consistent with results showing that production of ROS, which is negatively regulated by Nrf2, was also reduced at 24 h after hepatocytes were treated with GDN (FIG. 3B).

Next, using knock out and knock in strategies, it was determined whether shogaol in the GDN plays a role in the activation of Nrf2 in the context of lipids extracted from GDN. For knock-out, lipids of GDN without shogaol were carefully recovered from TLC silica gel plates, then liposome-like nanoparticles (LN) with shogaol knock-out were generated using previously described technology. For knock-in, commercial 6-shogaol was added to shogaol knock-out lipid to make knock-in liposome-like nanoparticles (FIG. 3C).

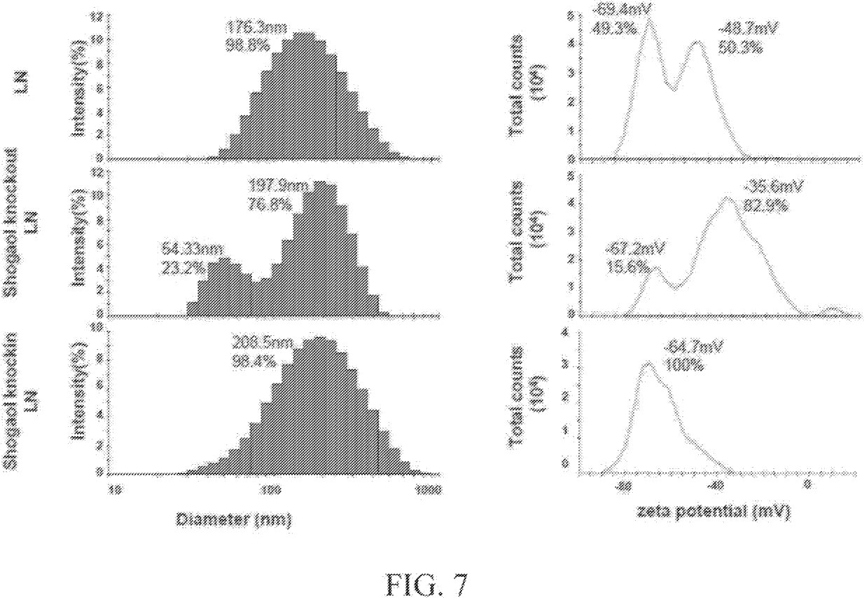

Although knock-out of shogaol in GDN derived LN has no effect on the range of size of reassembled nanoparticles, an additional subpopulation with a peak size of 54.33 nm was observed (FIG. 7). Knock-in of 6-shogaol led to elimination of this subpopulation. Zeta potential measurements indicated that LNs had a negative zeta potential value ranging from -76.2 mV to -33.5 mV (FIG. 7).

The effect(s) of knock-out of shogaol was further evaluated in terms of nuclear translocation of Nrf2. As the western blot analysis results for Nrf2 indicated, more nuclear Nrf2 was detected in the primary hepatocytes treated with GDN or GDN derived LN than in PBS treated hepatocytes (FIG. 3D). Moreover, knock-out of shogaol in GDN derived LN led to reduction of Nrf2 detected in the nucleus; whereas, knock-in of 6-shogaol resulted in the restoration of the levels of nuclear Nrf2 (FIG. 3E). This result was also consistent with results showing that production of ROS was also reduced 24 h after hepatocytes were treated with GDN or GDN derived LN with shogaol knock-in (FIG. 3F). Collectively, these data supported the conclusion that GDN shogaol has a role in increasing nuclear translocation of Nrf2 in GDN targeted hepatocytes.

Nrf2 is anti-inflammatory, as evidenced by the fact that Nrf2 KO mice have a tendency to develop autoimmune and inflammatory lesions in multiple tissues. The data from an in vitro cell culture study suggested that 6-shogaol suppressed LPS induced inflammation through Toll-like receptors (TLRs) mediated pathway. The TLRs are a major class of transmembrane proteins of the mammalian innate immune system and play a critical role in the inflammatory response. The major adaptors that bind to the intracellular domain of TLRs to activate the pro-inflammatory response are the myeloid differentiation primary response (MyD) 88 and TIR-domain-containing adapter-inducing interferon-0 (TRIF). Together, MyD88 and TRIF lead to the expression of numerous inflammatory factors through transcriptional factors such as NF-_{K0}, AP-1, and IRF-3 activation. Therefore, using hepatocytes from either MyD88 or TRIF knockout mice, the role of MyD88 and TRIF was determined in the GDN mediated activation of Nrf2. Western blot analysis indicated that primary hepatocytes from either TLR4 or TRIF knock-out mice had no increase in nuclear Nrf2 after stimulation with LN derived from GDN (100 µg/mL) or shogaol knock-in LN derived from GDN (100 µg/mL) when compared with cells treated with GDN and Shogaol KO LN (FIG. 3G). However, primary hepatocytes from Myd88 KO mice have no impairment in an increase in nuclear Nrf2 after stimulation with shogaol knock-in GDN derived LN (FIG. 3G, right panel) in comparison with stimulation with Shogaol KO LN. In summary, these results indicated that the TLR4/TRIF pathway plays a role in GDN shogaol-mediated activation of Nrf2 in mouse hepatocytes.

### Example 4 - Oral Administration of GDN Protects Mice From Alcohol Induced Liver Injury and Damage

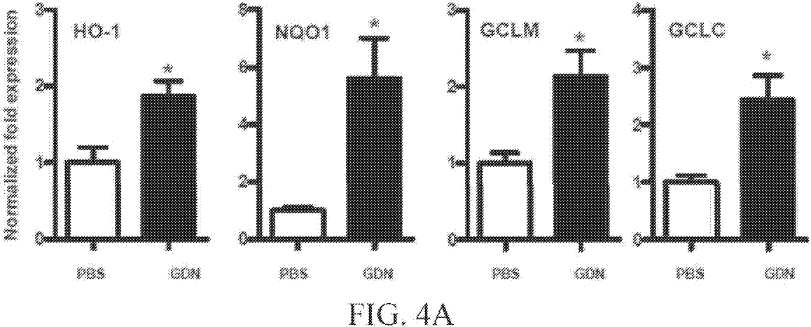

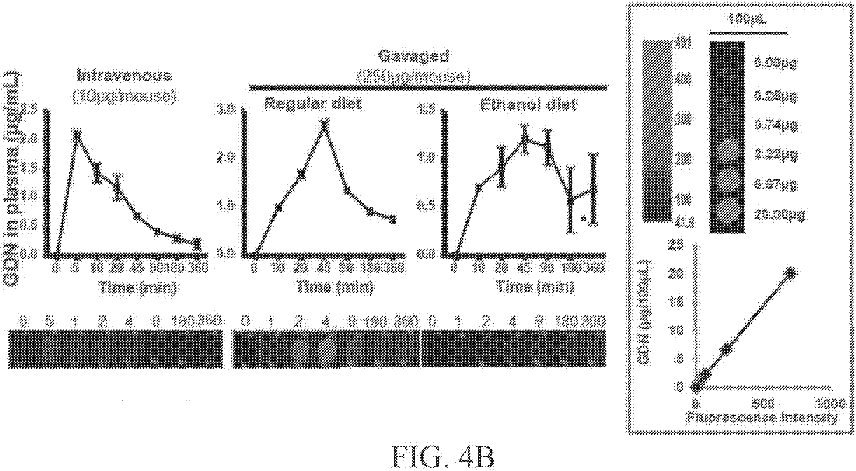

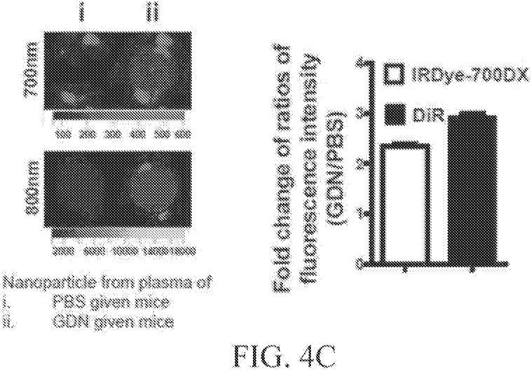

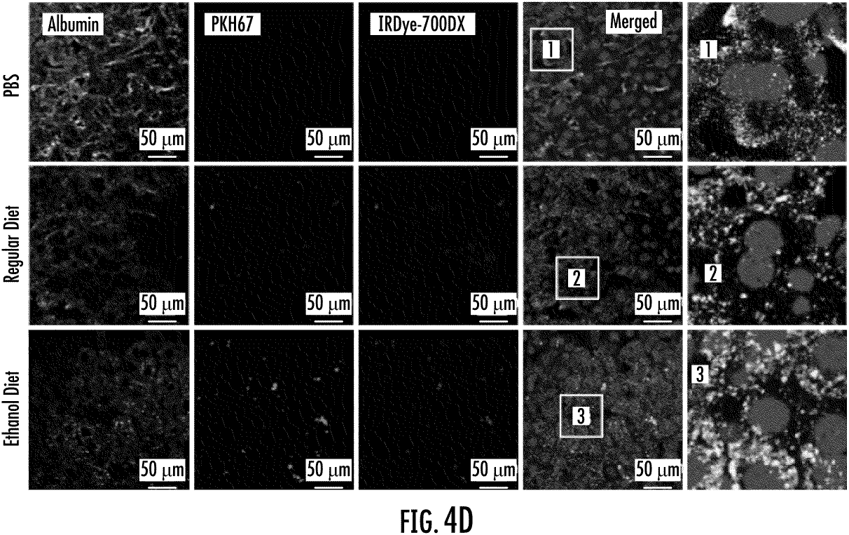

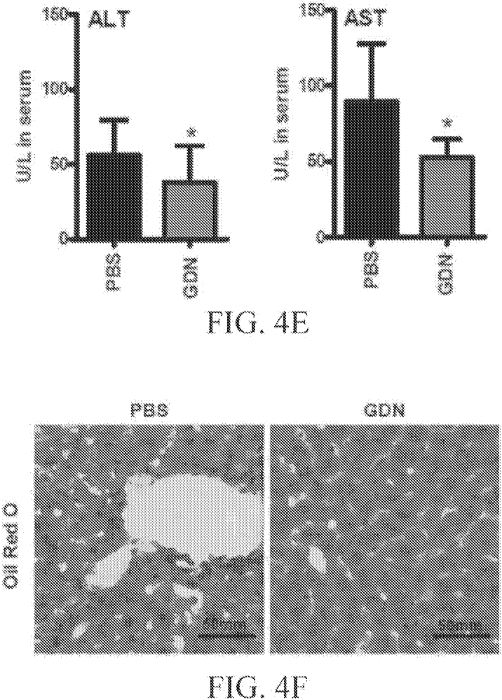

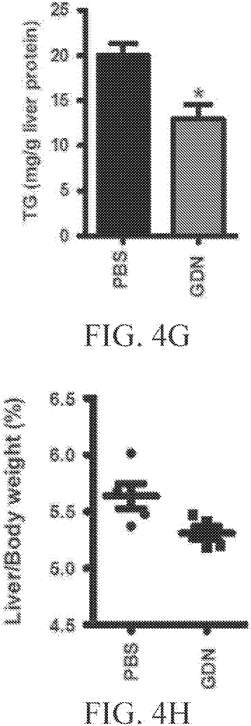

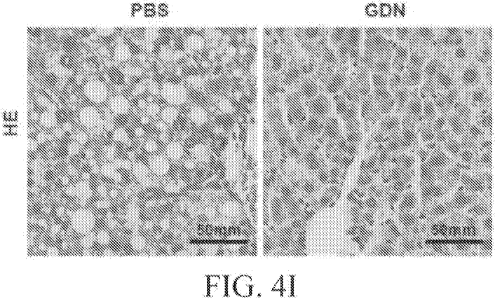

Ethanol-induced oxidative damage in the liver involves depletion of antioxidants. Real time PCR data support that a group of detoxifying/antioxidant genes including HO-1, NQO1, GCLM, and GCLC are induced in the liver 6 hr. after mice are orally administered GDN at a dose of 50 mg/mouse (FIG. 4A). To further determine how much GDN gets into the peripheral blood, mice were gavaged with IRDye-700DX covalent-conjugated GDN. Peripheral blood was then collected over time and circulating GDN was isolated using a standard protocol for isolation of exosomes. IRDye-700DX fluorescent signals of GDN in peripheral blood were then quantitatively analyzed. The results indicated that circulating GDN was detected 10 min after mice were gavaged with IRDye-700DX⁺GDN, reached a peak (2.8 µg/ml) at 45 min and then essentially returned to basal level 360 min after gavaging (FIG. 4B). In comparison with peak level of GDN given intravenously, it was estimated that approximately 5% of gavaged GDN gets into the peripheral blood. The fact of GDN getting into the peripheral blood was also confirmed with the data generated from IRDye-700DX⁺DIR⁺ double labeled GDN (FIG. 4C) purified from plasma. Interestingly, the levels of circulating GDN collected over time (all points) were lower in the blood of mice fed an ethanol diet when compared to mice fed a regular diet (FIG. 4B); whereas a higher level of IRDye-700DX⁺PKH67⁺GDN was detected in the liver of mice fed an alcoholic diet (FIG. 4D).

Mice that were pre-treated with vehicle only exhibited symptoms characteristic of alcohol-induced liver injury, including elevated levels of serum alanine aminotransferase (ALT) and aspartate aminotransferase (AST) (FIG. 4E). Histological analysis of the liver revealed accumulation of lipid droplets in the livers of ethanol-fed animals; whereas, lipid droplets were remarkably reduced in livers of the mice treated with GDN (FIG. 4F). In addition, compared with mice fed with alcohol alone, mice gavaged with GDN had significantly decreased the liver triglyceride levels (FIG. 4G) and liver weight (FIG. 4H). There were also histopathological changes in the liver (FIG. 4I) with extensive areas of typical fatty liver including macrovesicular steatosis in alcohol treated mice, and fat accumulation in hepatocytes, marked hemorrhage, and mononuclear cell infiltrates scattered diffusely throughout the viable parenchyma in comparison with the liver of mice gavage fed with GDN. Collectively, these results demonstrated that GDN treatment protected against the development of alcoholic liver injury in mice.

### Discussion of Examples 1-4

Characterization of ginger derived nanoparticles (GDNs). In this study, using a sucrose gradient centrifugation isolation method, ginger derived nanoparticles were isolated and purified from two sucrose bands (GDN and GDEN2). The nanoparticles were characterized for their morphology using atomic force microscopy (AFM) and were also characterized for charge and size. The criteria for naming mammalian cell derived nanoparticles have been established. Whether the same criteria can be applied to nanoparticles isolated from plant cells will require further study.

An in vivo distribution of orally administered GDN. Unlike the free form of shogaol which rapidly passes through gut after oral administration, the presently described results showed that the most of shogaol in ginger extract was not presented as a free form and was carried by GDN and target-delivered to hepatocytes. Therefore, a much less amount of shogaol carried by GDN than free form of shogaol may be required for having equal biological effect on the hepatocytes. Moreover, a person will eat more than one kind of food in a single meal thus consuming a variety of nanoparticles. These mixed nanoparticles with different biological activities may have different biological effects on the same recipient cells or be taken up by different types of cells and subsequently work in a coordinated manner to maintain tissue homeostasis. Here, the biological effect of ginger nanoparticles that was demonstrated on the targeted cells, i.e., hepatocytes, prevented alcoholic induced damage, whereas grapefruit derived nanoparticles were taken up by Kupffer cells. Both hepatocytes and Kupffer cells play a critical role in liver homeostasis. If a person takes both in the same meal, it is conceivable that better liver homeostasis could be achieved. Also, most of diseases end up with multiple cells functionally dysregulated. These studies may lead to the design of customized or personalized cocktails of edible nanoparticles from different plants that may target to different types of cells in the liver and have different biological effects. Therefore, better preventative and therapeutic outcomes are contemplated.

In the above-described study, it was also demonstrated that although both GDN and GDEN2 were taken up by hepatocytes, different pathways were utilized. It was shown that nanoparticles can enter the cells by endocytosis. Endocytosis consists of three major steps: formation of membrane vesicles with the cargo, endosomal delivery of the cargo inside the cell, and the distribution to various organelles inside the cell. Endocytosis, in general, is routinely distinguished from clathrin-mediated endocytosis, caveolin-mediated endocytosis, clathrin- or caveolin-independent endocytosis, and macropinocytosis. The present data indicate that GDN is taken up through macropintocytosis as indicated by the fact that nontoxic concentrations of amiloride in hepatocytes inhibit macropinocytosis. In contrast, treatment with a microtubule-disrupting agent (nocodazole) indicated that GDEN2 was taken up predominantly through microtubule-dependent active transport.

In general, the quantity of nanoparticles getting into the peripheral blood is a parameter for evaluating the stability of food derived nanoparticles. Here, a double labeling GDN procedure was used for keeping track of GDN. The NHS ester reactive group of IRDye-700DX was covalently conjugated with primary and secondary amines in GDN proteins; whereas DIR binds to GDN lipids. It was able to be shown that about 5% of the IRDye-700DX⁺GDN gets into the peripheral blood in comparison with an intravenous injection of GDN as a reference.

Activation of nuclear factor erythroid-2-relatedfactor-2 (Nrf2) is dependent on GDN 6-shogaol through TLR4/TRIF pathway. Using knock out and knock in strategy, it was identified that shogaol in the GDN plays a role in the induction of Nrf2 nuclear translation in the targeted hepatocytes. Using a standard technology for characterization of extracellular microvesicles, it was demonstrated that hepatocyte incorporation of GDN leads to Nrf2 nuclear translocation. The data also show that the TLR4/TRIF pathway plays a role in GDN mediated Nrf2 nuclear translocation activity. Using knock-out and knock-in strategies, it was further demonstrated that GDN shogaol plays a role in induction of Nrf2 nuclear translocation.

Nrf2 nuclear translocation leads to activation of a pleiotropic cytoprotective defense process that includes antioxidants and protects against inflammatory disorders by inhibiting oxidative tissue injuries. The real-time PCR data supported the finding that a number of Nrf2 regulated genes encoding for cytoprotective defenses of hepatocytes stimulated by GDN were upregulated. Using knock-out and knock-in strategies utilizing shoagaol from GDN, it was clearly shown that shogaol plays a role in induction of Nrf2. The data also was supported by published data indicating that shogaol rich extract from ginger induces Nrf2 nuclear translocation. It was also conceivable that intact ginger derived nanoparticles were likely to be required for in vivo targeted delivery of shogaol to hepatocytes. The finding that hepatocytes are specifically targeted by GDN (as shown in the foregoing study) indicated that delivery of therapeutic agents to hepatocytes without causing non-specific toxicity is feasible. In addition, using the knock-out and knock-in strategies as described above provides a means of identifying the role of each individual lipid in the complex of liposome-like nanoparticles.

Recent studies report crosstalk between Nrf2/HO-1 and the TLR system as a mechanism involved in hepatic injury. The presently described results indicated that GDN regulated the Nrf2 activity through the TLR4/TRIF pathway in hepatocytes. The TLR4/TRIF mediated pathway plays a crucial role in mammalian innate immune responses against pathogens and a variety of insults. The innate immune system is the dominant immune system found in plants, fungi, insects, and in primitive multicellular organisms. This finding provides a foundation for further determining whether edible plant derived nanoparticles may also regulate innate immune response in plant kingdoms.

Oral administration of GDN protects mice from alcohol induced liver damage. A number of studies indicate that the plant kingdom provides not only nutrients but bioactive natural products to prevent diseases that occur in the mammalian kingdom. How plant derived bioactive natural products execute their functions on the mammalian system while resisting the harsh GI environment, i.e., the low pH in the stomach and the degradative enzymes, and more importantly, how plant derived bioactive natural products which are a foreign material to the mammalian immune system are ignored by immune cells is not well understood. Oral administration of GDN leads to protection of mice from alcohol-induced liver injury. This finding not only indicates that GDN could be used as a novel agent to protect the liver against damage, but provides a foundation for studying the mechanism underlying interspecies communication through nanoparticles individuals ingest daily from many different types of edible plants.

Throughout this document, various references are mentioned. All such references are incorporated herein by reference, including the references set forth in the following list:

### REFERENCES

1. Ju S, Mu J, Dokland T, Zhuang X, Wang Q, Jiang H, et al. Grape exosome-like nanoparticles induce intestinal stem cells and protect mice from DSS-induced colitis. Mol Ther. 2013;21(7): 1345-57.

2. Mu J, Zhuang X, Wang Q, Jiang H, Deng ZB, Wang B, et al. Interspecies communication between plant and mouse gut host cells through edible plant derived exosome-like nanoparticles. Molecular nutrition & food research. 2014;58(7):1561-73.

3. Raimondo S, Naselli F, Fontana S, Monteleone F, Lo Dico A, Saieva L, et al. Citrus limon-derived nanovesicles inhibit cancer cell proliferation and suppress CML xenograft growth by inducing TRAIL-mediated cell death. Oncotarget. 2015;6(23):19514-27.

4. Bak MJ, Ok S, Jun M, Jeong WS. 6-shogaol-rich extract from ginger up-regulates the antioxidant defense systems in cells and mice. Molecules. 2012;17(7):8037-55.

5. Lu Y, Zhang XH, Cederbaum AI. Ethanol induction of CYP2A5: role of CYP2E1-ROS-Nrf2 pathway. Toxicol Sci. 2012;128(2):427-38.

6. Lee HG, Li MH, Joung EJ, Na HK, Cha YN, Surh YJ. Nrf2-Mediated heme oxygenase-1 upregulation as adaptive survival response to glucose deprivation-induced apoptosis in HepG2 cells. Antioxid Redox Signal. 2010;13(11):1639-48.

7. Beyer TA, Xu W, Teupser D, auf dem Keller U, Bugnon P, Hildt E, et al. Impaired liver regeneration in Nrf2 knockout mice: role of ROS-mediated insulin/IGF-1 resistance. Embo J. 2008;27(1):212-23.

8. Wang B, Zhuang X, Deng ZB, Jiang H, Mu J, Wang Q, et al. Targeted drug delivery to intestinal macrophages by bioactive nanovesicles released from grapefruit. Mol Ther. 2013.

9. Johnson DA, Amirahmadi S, Ward C, Fabry Z, Johnson JA. The absence of the pro-antioxidant transcription factor Nrf2 exacerbates experimental autoimmune encephalomyelitis. Toxicological sciences : an official journal of the Society of Toxicology. 2010;114(2):237-46.

10. Yoh K, Itoh K, Enomoto A, Hirayama A, Yamaguchi N, Kobayashi M, et al. Nrf2-deficient female mice develop lupus-like autoimmune nephritis. Kidney international. 2001;60(4): 1343-53.

11. Park SH, Kyeong MS, Hwang Y, Ryu SY, Han SB, Kim Y. Inhibition of LPS binding to MD-2 co-receptor for suppressing TLR4-mediated expression of inflammatory cytokine by 1-dehydro-10-gingerdione from dietary ginger. Biochem Biophys Res Commun. 2012;419(4):735-40.

12. Ahn SI, Lee JK, Youn HS. Inhibition of homodimerization of toll-like receptor 4 by 6-shogaol. Mol Cells. 2009;27(2):211-5.

13. Gould SJ, Raposo G. As we wait: coping with an imperfect nomenclature for extracellular vesicles. Journal of extracellular vesicles. 2013;2.

14. Treuel L, Jiang X, Nienhaus GU. New views on cellular uptake and trafficking of manufactured nanoparticles. J R Soc Interface. 2013;10(82):20120939.

15. Rehman Z, Zuhorn IS, Hoekstra D. How cationic lipids transfer nucleic acids into cells and across cellular membranes: recent advances. Journal of controlled release : official journal of the Controlled Release Society. 2013;166(1):46-56.

16. Xu S, Olenyuk BZ, Okamoto CT, Hamm-Alvarez SF. Targeting receptor-mediated endocytotic pathways with nanoparticles: rationale and advances. Adv Drug Deliv Rev. 2013;65(1):121-38.

17. Deng ZB, Zhuang X, Ju S, Xiang X, Mu J, Liu Y, et al. Exosome-like nanoparticles from intestinal mucosal cells carry prostaglandin E2 and suppress activation of liver NKT cells. Journal of immunology. 2013;190(7):3579-89.

18. Wang R, Paul VJ, Luesch H. Seaweed extracts and unsaturated fatty acid constituents from the green alga Ulva lactuca as activators of the cytoprotective Nrf2-ARE pathway. Free Radic Biol Med. 2013;57:141-53.

19. Wu KC, Cui JY, Klaassen CD. Effect of graded Nrf2 activation on phase-I and -II drug metabolizing enzymes and transporters in mouse liver. PLoS One. 2012;7(7):e39006.

20. Kaspar JW, Niture SK, Jaiswal AK. Antioxidant-induced INrf2 (Keap1) tyrosine 85 phosphorylation controls the nuclear export and degradation of the INrf2-Cul3-Rbxl complex to allow normal Nrf2 activation and repression. J Cell Sci. 2012;125(Pt 4): 1027-38.

21. Jyrkkanen HK, Kansanen E, Inkala M, Kivela AM, Hurttila H, Heinonen SE, et al. Nrf2 regulates antioxidant gene expression evoked by oxidized phospholipids in endothelial cells and murine arteries in vivo. Circ Res. 2008;103(1):e1-9.

22. Kang JW, Lee SM. Melatonin inhibits type 1 interferon signaling of toll-like receptor 4 via heme oxygenase-1 induction in hepatic ischemia/reperfusion. Journal of pineal research. 2012;53(1):67-76.

23. Vandenbon A, Teraguchi S, Akira S, Takeda K, Standley DM. Systems biology approaches to toll-like receptor signaling. Wiley Interdiscip Rev Syst Biol Med. 2012;4(5):497-507.

24. Kawai T, Akira S. Pathogen recognition with Toll-like receptors. Curr Opin Immunol. 2005;17(4):338-44.

25. Medzhitov R, Janeway C, Jr. Innate immune recognition: mechanisms and pathways. Immunol Rev. 2000; 173:89-97.

26. Magrone T, Jirillo E. The interplay between the gut immune system and microbiota in health and disease: nutraceutical intervention for restoring intestinal homeostasis. Current pharmaceutical design. 2013;19(7):1329-42.

27. Crispe IN. The liver as a lymphoid organ. Annual review of immunology. 2009;27:147-63.

28. Alonso C, Vicario M, Pigrau M, Lobo B, Santos J. Intestinal barrier function and the brain-gut axis. Advances in experimental medicine and biology. 2014;817:73-113.

29. Faria AM, Gomes-Santos AC, Goncalves JL, Moreira TG, Medeiros SR, Dourado LP, et al. Food components and the immune system: from tonic agents to allergens. Frontiers in immunology. 2013;4:102.

30. Mann ER, Landy JD, Bernardo D, Peake ST, Hart AL, Al-Hassi HO, et al. Intestinal dendritic cells: their role in intestinal inflammation, manipulation by the gut microbiota and differences between mice and men. Immunology letters. 2013;150(1-2):30-40.

31. Jacobi SK, Odle J. Nutritional factors influencing intestinal health of the neonate. Advances in nutrition. 2012;3(5):687-96.

32. Kayama H, Takeda K. Regulation of intestinal homeostasis by innate and adaptive immunity. International immunology. 2012;24(11):673-80.

33. Vitetta L, Briskey D, Hayes E, Shing C, Peake J. A review of the pharmacobiotic regulation of gastrointestinal inflammation by probiotics, commensal bacteria and prebiotics. Inflammopharmacology. 2012;20(5):251-66.

34. Magnusson MK, Wick MJ. Intestinal dendritic cell and macrophage subsets: Tipping the balance to Crohn’s disease? European journal of microbiology & immunology. 2011; 1(1): 19-24.

35. Peron JP, de Oliveira AP, Rizzo LV. It takes guts for tolerance: the phenomenon of oral tolerance and the regulation of autoimmune response. Autoimmunity reviews. 2009;9(1):1-4.

36. Zhang L, Hou D, Chen X, Li D, Zhu L, Zhang Y, et al. Exogenous plant MIR168a specifically targets mammalian LDLRAP1: evidence of cross-kingdom regulation by microRNA. Cell research. 2012;22(1): 107-26.

37. Sala-Cirtog M, Marian C, Anghel A. New insights of medicinal plant therapeutic activity-The miRNA transfer. Biomedicine & pharmacotherapy = Biomedecine & pharmacotherapie. 2015;74:228-32.

38. Regente M, Pinedo M, Elizalde M, de la Canal L. Apoplastic exosome-like vesicles: a new way of protein secretion in plants? Plant signaling & behavior. 2012;7(5):544-6.

39. Ding Y, Wang J, Wang J, Stierhof YD, Robinson DG, Jiang L. Unconventional protein secretion. Trends in plant science. 2012;17(10):606-15.

40. Wang B, Zhuang X, Deng ZB, Jiang H, Mu J, Wang Q, et al. Targeted drug delivery to intestinal macrophages by bioactive nanovesicles released from grapefruit. Molecular therapy : the journal of the American Society of Gene Therapy. 2014;22(3):522-34.

41. Bligh EG, Dyer WJ. A rapid method of total lipid extraction and purification. Can J Biochem Physiol. 1959;37(8):911-7.

42. Wanjie SW, Welti R, Moreau RA, Chapman KD. Identification and quantification of glycerolipids in cotton fibers: reconciliation with metabolic pathway predictions from DNA databases. Lipids. 2005;40(8):773-85.

43. Torrelles JB, Sieling PA, Zhang N, Keen MA, McNeil MR, Belisle JT, et al. Isolation of a distinct Mycobacterium tuberculosis mannose-capped lipoarabinomannan isoform responsible for recognition by CDlb-restricted T cells. Glycobiology. 2012;22(8): 1118-27.

44. Guo P. RNA Nanotechnology: methods for synthesis, conjugation, assembly and application of RNA nanoparticles. Methods. 2011;54(2):201-3.

45. Hermida LG S-XM, Barnadas-Rodriguez R. Combined strategies for liposome characterization during in vitro digestion. Journal of Liposome Research. 2009; 19(3):207-19.

46. Liu Y, Shah SV, Xiang X, Wang J, Deng ZB, Liu C, et al. COP9-associated CSN5 regulates exosomal protein deubiquitination and sorting. Am J Pathol. 2009;174(4): 1415-25.

47. Sun D, Zhuang X, Xiang X, Liu Y, Zhang S, Liu C, et al. A novel nanoparticle drug delivery system: the anti-inflammatory activity of curcumin is enhanced when encapsulated in exosomes. Mol Ther. 2010;18(9):1606-14.

48. Zhuang X, Deng Z, Mu J, Zhang L, Yan J, Miller D, Feng W, McClain C, Zhang HG. Ginger-derived nanoparticles protect against alcohol-induced liver damage. J Extracellular Vesicles. 2015; 4: 10.3402/jev.v4.28713.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for treating alcohol induced liver injury, comprising administering to a subject a preparation comprising ginger-derived nanoparticles, wherein the ginger-derived nanoparticles comprise an effective amount of a shogaol to treat the alcohol induced liver injury.

2. The method of claim 1, wherein the preparation is administered orally.

3. The method of claim 1, wherein the ginger-derived nanoparticles have an average diameter of about 100 nm to about 1000 nm.

4. The method of claim 1, wherein the ginger-derived nanoparticle comprises a phosphatidic acid (PA), a digalactosyldiacylglycerol (DGDG), a monogalactosyl monoacylglycerol (MGMG), and combinations thereof.

5. The method of claim 1, wherein administering the preparation increases an amount of nuclear factor erythroid-2 related factor (Nrf2) activation.

6. The method of claim 1, wherein the preparation is administered prior to contacting a liver cell of a subject with an amount of alcohol.

7. A method for decreasing nuclear factor erythroid-2 related factor (Nrf2) activation in a hepatocyte, comprising contacting the hepatocyte with a preparation comprising ginger-derived nanoparticles.

8. The method of claim 7, wherein the ginger-derived nanoparticles have an average diameter of about 300 nm to about 400 nm.

9. A method for increasing nuclear factor erythroid-2 related factor (Nrf2) activation in a hepatocyte, comprising contacting the hepatocyte with a preparation, optionally a pharmaceutical composition, comprising ginger-derived nanoparticles in an amount and via a route sufficient to increase Nrf2 activation in the hepatocyte.

10. The method of claim 9, wherein the ginger-derived nanoparticle is administered orally.

11. The method of claim 9, wherein the ginger-derived nanoparticle has an average diameter of about 100 nm to about 1000 nm, optionally an average diameter of about 300 nm to about 400 nm.

12. The method of claim 9, wherein the ginger-derived nanoparticle comprises a phosphatidic acid (PA), a digalactosyldiacylglycerol (DGDG), a monogalactosyl monoacylglycerol (MGMG), and combinations thereof.

13. The method of claim 12, wherein the ginger-derived nanoparticle comprises a PA content of about 30% to about 40%, a DGDG content of about 30% to about 40%, and an MGMG content of about 10% to about 20%.

14. The method of claim 9, wherein the ginger-derived nanoparticles comprise an effective amount of a shogaol, optionally 6-shogaol, to increase Nrf2 activation in the hepatocyte.

15. The method of claim 9, wherein the preparation is a pharmaceutical composition formulated for oral administration.
